SEQUENCE LISTING (22 sequences)

SEQ ID NO: 1 - DNA, 102 bp, Artificial Sequence
  Fully synthetic
ctgggaaaactacggtgagcatatgtttaccacttcttcagagaaccgtgaatattgcgttaaaccgatgaactgcccgggttacgccccgccctgccactc

SEQ ID NO: 2 - DNA, 108 bp, Artificial Sequence
  Fully synthetic
aaatccatgattaccaacccactcccaaagatattataaaatctcaacatgctgatttgatcctttggaatgggctaaatctagaggtaacgaatcagacaattgacg

SEQ ID NO: 3 - DNA, 101 bp, Artificial Sequence
  Fully synthetic
agatgacgccctctccactctgcataccaattccacataaccactcttcaaattataatgctcacagaccctctaaggttttacgccccgccctgccactc

SEQ ID NO: 4 - DNA, 103 bp, Artificial Sequence
  Fully synthetic
catagtgacccattgttatagagtacttagctatgcctcagagtaatgctcttgatctaggtgctttctttttacttgttggtaacgaatcagacaattgacg

SEQ ID NO: 5 - DNA, 106 bp, Artificial Sequence
  Fully synthetic
ggattttataagtgaagggtgacatttgtagtattccttaccccgtctggttcctcatacgcattaactcataatccttggtaccttacgccccgccctgccactc

SEQ ID NO: 6 - DNA, 104 bp, Artificial Sequence
  Fully synthetic
tgccctttcagaagacctaggtttgattcctagcacccactgatgctaacaaccagacataatgccagttctaggggatccggtaacgaatcagacaattgacg

SEQ ID NO: 7 - DNA, 101 bp, Artificial Sequence
  Fully synthetic
gtttaactcccttaacacacacacacacacatgcacacgcacatgcacacgcgcactcacaccacaaaatcacacaatttttacgccccgccctgccactc

SEQ ID NO: 8 - DNA, 103 bp, Artificial Sequence
  Fully synthetic
ctgtgctcagagctttgagtgccctacatataacaggacacaaactcttccttactttctgattgccacatggtccatttggtaacgaatcagacaattgacg

SEQ ID NO: 9 - DNA, 101 bp, Artificial Sequence
  Fully synthetic
ctatgccataggaatttggaggaaattaggcttactggtccattaattatttccaacggatgcagtcagaatgtatatttttacgccccgccctgccactc

SEQ ID NO: 10 - DNA, 103 bp, Artificial Sequence
  Fully synthetic
ttagagcagacagtgctcttaaccgttgagccatctctccagtccctggacacactgattttaagttgacattaaaatttggtaacgaatcagacaattgacg

SEQ ID NO: 11 - DNA, 102 bp, Artificial Sequence
  Fully synthetic
ccaccacgccaggctaattttagcttaataccctattacctctcaggtgtgtctaaatttttctttgatggaaaaactagtttacgccccgccctgccactc

SEQ ID NO: 12 - DNA, 104 bp, Artificial Sequence
  Fully synthetic
agttatgtcagtttgctattataatgtttattattattccttgaataaaaaagaatatgcacctaataaacgttgactagtggtaacgaatcagacaattgacg

SEQ ID NO: 13 - DNA, 107 bp, Artificial Sequence
  Fully synthetic
agtggccctagagggtcggtaaggagccaagggcctatgcaaggatgctcacacacctcccatccccaccttcccagacacatatgttacgccccgccctgccactc

SEQ ID NO: 14 - DNA, 103 bp, Artificial Sequence
  Fully synthetic
gtgctgtccaagcttggctcatctggggtttgctgggcttaacacccaataaagaactttgctgactactaagcccagtaggtaacgaatcagacaattgacg

SEQ ID NO: 15 - DNA, 100 bp, Artificial Sequence
  Fully synthetic
acggccggcgactgctcctcagtgaggaggcgtcactcaatatccctgcagtggcggccgcccatgtgatcaaacggtattacgccccgccctgccactc

SEQ ID NO: 16 - DNA, 103 bp, Artificial Sequence; identical to SEQ ID NO: 14
  Fully synthetic

SEQ ID NO: 17 - DNA, 101 bp, Artificial Sequence
  Fully synthetic
cctagagggtcggtaaggagccaagggcctatgcaaggatgctcacacacctcccatccccaccttcccagacacatatgttacgccccgccctgccactc

SEQ ID NO: 18 - DNA, 103 bp, Artificial Sequence
  Fully synthetic
aagaactagactagaagttggaaaacaggtttcactgcatcccttcacagtgaatggcccttctcttcagcactcatatgggtaacgaatcagacaattgacg

SEQ ID NO: 19 - DNA, 71 bp, Artificial Sequence
  Fully synthetic
tcctagcatcttatcttttaaaaaacaaggcgcagtttgaagatcgaaatgaagtgcataccaatcaggac

SEQ ID NO: 20 - DNA, 89 bp, Artificial Sequence
  Fully synthetic
atcatgtaaatctacagtagcaaccaaatgtttcttctcttcccaagaaactatttagtcaagcgctcttgtcgtcgtcatccttgtag

SEQ ID NO: 21 - DNA, 71 bp, Artificial Sequence
  Fully synthetic
ttctagcatcttatcttttaaaaaacaaggcacagtttgaagatcgaaacgaagtgcataccaatcaggac

SEQ ID NO: 22 - DNA, 89 bp, Artificial Sequence
  Fully synthetic
atcatgtaaatctacagtagcaactaaatttttctgttcttcccattaagctatttagtcaagcgctcttgtcgtcgtcatccttgtag

(19) United States
(12) Patent Application Publication
(10) Pub. No.: US 2020/0010867 A1
(43) Pub. Date: Jan. 9, 2020

(54) METHOD OF HOMOLOGOUS RECOMBINATION OF DNA

(71) Applicants: SHANDONG UNIVERSITY, QINGDAO (CN); TECHNISCHE UNIVERSITÄT DRESDEN, DRESDEN (DE)

(72) Inventors: Youming ZHANG, Jinan (CN); Hailong WANG, Jinan (CN); Jun FU, Jinan (CN); Adrian Francis STEWART, Dresden (DE)

(21) Appl. No.: 16/578,385

(22) Filed: Sep. 22, 2019

Related U.S. Application Data
(63) Continuation-in-part of application No. PCT/CN2017/000483, filed on Aug. 2, 2017.

(30) Foreign Application Priority Data
Mar. 23, 2017 (CN) 201710177676.1

(51) Int. Cl. C12P 19/34; C12N 15/90; C12N 9/22

(52) U.S. Cl. CPC C12P 19/34; C12N 9/22; C12N 15/907

(57) ABSTRACT

A method of homologous recombination, including in vitro joining two or more target nucleic acid molecules with a first exonuclease, and recombining the two or more target nucleic acid molecules in the presence of a second exonuclease and an annealing protein. The recombined target nucleic acid molecules share at least one homologous sequence.

### CROSS-REFERENCE TO RELATED APPLICATIONS

This application is a continuation-in-part of International Patent Application No. PCT/CN2017/000483 with an international filing date of Aug. 2, 2017, designating the United States, now pending, and further claims foreign priority benefits to Chinese Patent Application No. 201710177676.1 filed Mar. 23, 2017. The contents of all of the aforementioned applications, including any intervening amendments thereto, are incorporated herein by reference.

### BACKGROUND

The disclosure relates to a method of cloning and assembly of deoxyribonucleic acid (DNA).

Conventionally, short DNA fragments are obtained by chemical synthesis and amplified by PCR, while cloning of DNA fragments larger than 10-kb depends on the construction and screening of DNA libraries. Conventional methods of library construction and screening are time-consuming and laborious, and the target DNA fragments are often located on several different clones.

Large DNA fragments larger than 10-kb can also be assembled from small fragments using Gibson assembly in vitro or DNA assembler in vivo. However, in the assembly process, small fragments tend to mutate randomly.

Direct cloning refers to cloning a specific DNA sequence of a genomic DNA into a vector. The process involves recombinant proteins RecE and RecT. However, RecET direct cloning fails to synthesize DNA fragments larger than 50 kb from a bacterial genome or clone DNA fragments larger than 10 kb from the mammalian genome. In addition, RecET direct cloning can only synthesize up to five DNA fragments.

### SUMMARY

In one aspect, the disclosure provides a method of homologous recombination, the method comprising in vitro joining two or more target nucleic acid molecules with a first exonuclease, and recombining the two or more target nucleic acid molecules in the presence of a second exonuclease and an annealing protein, wherein recombined target nucleic acid molecules share at least one homologous sequence.

In another aspect, the disclosure provides a method of homologous recombination, the method comprising in vitro joining a first nucleic acid molecule and a second nucleic acid molecule in the presence of a first exonuclease, and recombining the first nucleic acid molecule and the second nucleic acid molecule in the presence of a second exonuclease and an annealing protein, wherein the first nucleic acid molecule and the second nucleic acid molecule share at least one homologous sequence.

In still another aspect, the disclosure provides a method of assembling a linear nucleic acid molecule, the method comprising in vitro joining two or more nucleic acid molecules with a first exonuclease, and recombining the two or more nucleic acid molecules in the presence of a second exonuclease and an annealing protein, wherein each nucleic acid molecule shares at least one homologous sequence with an adjacent nucleic acid molecule in a resulting assembly product.

Also, the disclosure provides a method of cloning genomic DNA, the method comprising in vitro joining a linear cloning vector and a mixture of genomic DNA fragments with a first exonuclease, and recombining the linear cloning vector and a target DNA fragment of the mixture of genomic DNA fragments, wherein the linear cloning vector shares at least one homologous sequence with the target DNA fragment of the mixture of genomic DNA fragments.

The at least one homologous sequence can be in or at one end of the two or more target nucleic acid molecules, particularly, at least one homologous sequence at one end of a target nucleic acid molecule, and more particularly, all the homologous sequences at one end of the target nucleic acid molecules.

The at least one homologous sequence has a length of at least 6, at least 10, at least 20, at least 25, at least 30, at least 40, at least 50, at least 60, at least 70, at least 80 nucleotides, particularly 25, 40 or 80 nucleotides, and more particularly 80 nucleotides.

The first exonuclease can be a 5′ to 3′ exonuclease or a 3′ to 5′ exonuclease, particularly T4 DNA polymerase, Klenow fragment of DNA polymerase I, T5 exonuclease, T7 exonuclease, and more particularly T4 DNA polymerase or T5 exonuclease.

In vitro joining in the presence of the first exonuclease comprises cleaving and annealing. The in vitro joining can join two or more target nucleic acid molecules or the first nucleic acid molecule to the second nucleic acid molecule, or join the treated linear cloning vector to the target DNA fragment of a mixture of genomic DNA fragments.

In vitro joining in the presence of the first exonuclease comprises enzyme digestion and annealing; the enzyme digestion of different nucleic acid molecules can be performed separately or simultaneously, such as in a mixture of samples.

In vitro joining in the presence of the first exonuclease further comprises addition of a DNA polymerase, dNTPs and a DNA ligase.

In vitro joining in the presence of the first exonuclease further comprises addition of a DNA polymerase having 3′ to 5′ exonuclease activity.

In vitro joining in the presence of the first exonuclease further excludes the addition of dNTPs.

In vitro joining in the presence of the first exonuclease further comprises T4 DNA polymerase treatment or Gibson assembly.

The second exonuclease is RecE, and particularly, the RecE is a recombinant expression product.

The annealing protein includes RecA, RAD51, Redβ, RecT, Pluβ or RAD52, and particularly, the annealing protein is RecT, more particularly, the RecT is a recombinant expression product.

The annealing protein is RecT, particularly, the RecT is a recombinant expression product.

The homologous recombination is carried out in vitro or in a host cell.

The host cell can be a yeast cell, particularly the yeast cell is a Saccharomyces cerevisiae cell; or a bacterial cell, particularly the bacterial cell is Bacillus subtilis or Escherichia coli.

The host cell expresses an exonuclease, particularly a second exonuclease and an annealing protein.

The host cell expresses an exonuclease, an annealing protein, and Redγ. Particularly, the host cell further expresses RecA, more particularly, the host cell expresses RecE, RecT, Redγ, and RecA.

The host cell is E. coli cell expressing full length RecE and/or RecT, particularly, the host cell is E. coli cell expressing full length RecE, RecT and Redγ, more particularly the host cell is E. coli cell expressing full length RecE, RecT, Redγ and RecA.

The host cell is E. coli cell expressing truncated RecE and RecT.

The host cell is E. coli cell expressing Redα and Redβ.

The host cell expresses an exonuclease on a plasmid vector and/or chromosome, particularly, the second exonuclease, the annealing protein, Redγ and/or RecA, particularly, expressing by a plasmid vector, and more particularly, expressing by a plasmid vector and a chromosome simultaneously.

The target nucleic acid molecule or the target DNA fragment is a linear DNA segment selected from a DNA fragment digested by endonuclease, a DNA fragment amplified by PCR, a genomic DNA fragment, a member of cDNA library, a fragment derived from bacterial artificial chromosomes (BACs), and a fragment of cloning vectors.

The endonuclease can be a restriction enzyme or a programmable endonuclease, such as Cas9.

The number of the target nucleic acid molecules or DNA fragments is 2, 3, 4, 5, 6, 7, 8, 9, 10, 11, 12, 13, 14, 15, 16, 17, 18, 19, 20 or more.

The target nucleic acid molecule comprises a sequence of 0.5 kb or longer (e.g., 1-kb or longer, 2.5-kb or longer, 4-kb or longer, 5-kb or longer, 7.5-kb or longer, 10-kb or longer, 15-kb or longer, 20-kb or longer, 25-kb or longer, 40-kb or longer, 50-kb or longer, 75-kb or longer or 100-kb or longer).

The two or more target nucleic acid molecules, the first target nucleic acid molecule, and the second target nucleic acid molecule or target DNA fragments comprise one or more PCR amplified DNA fragments, genomic DNA fragments, cDNA library members, and/or a fragment derived from BAC.

The first exonuclease has 3′ to 5′ exonuclease activity, and particularly, is T4 DNA polymerase.

In vitro joining in the presence of a first exonuclease is carried out in vitro in the absence of dNTPs.

The second exonuclease is a full length RecE.

The annealing protein is RecT.

The homologous recombination is carried out in a bacterial host cell expressing the full-length RecE and RecT, particularly in Escherichia coli.

The two or more target nucleic acid molecules comprise one or more PCR amplified DNA fragments, genomic DNA fragments, cDNA library members, and/or a fragment derived from BAC, linear plasmid and/or cloning vector fragment, and particularly, three or more linear plasmids and/or cloning vector fragments.

The first exonuclease comprises a Gibson assembly.

The second exonuclease is full length RecE.

The annealing protein is RecT.

The homologous recombination is carried out in a bacterial host cell expressing the full length RecE and RecT, particularly in E. coli.

The two or more target nucleic acid molecules include three or more PCR amplified DNA fragments, genomic DNA fragments, cDNA library members, and/or a fragment derived from BAC, linear plasmids and/or cloning vector fragments, particularly three or more linear plasmids and/or cloning vector fragments.

The first exonuclease comprises a Gibson assembly.

The second exonuclease is full length RecE.

The annealing protein is RecT.

The homologous recombination is carried out in a bacterial host cell expressing the full length RecE and RecT, particularly in E. coli.

Also provided is a kit comprising the first exonuclease and the second exonuclease described in the preceding method, or a nucleic acid encoding the first exonuclease and the second exonuclease described in the preceding method.

A kit comprises the first exonuclease and the second exonuclease described in the preceding method, or a nucleic acid encoding the first exonuclease and the second exonuclease. Particularly, the kit further comprises a host cell expressing the second exonuclease, particularly, the host cell expresses an exonuclease, an annealing protein and Redγ, particularly, the host cell also expresses RecA, more particularly, the host cell expresses RecE, RecT, Red γ and RecA, the host cell can be a yeast cell, particularly the yeast cell is a Saccharomyces cerevisiae cell; or a bacterial cell, particularly the bacterial cell is Bacillus subtilis or Escherichia coli, The host cell expresses an exonuclease, an annealing protein, Redγ and/or RecA on a plasmid vector and/or chromosome simultaneously, particularly, expressed by a plasmid vector, the more particularly, expressed by a plasmid vector or chromosome, further particularly, the kit may further comprise one or more pre-prepared linear vectors.

The first exonuclease is a DNA polymerase having 3′ to 5′ exonuclease activity, such as T4 DNA polymerase, Klenow fragments of DNA polymerase I, T5 exonuclease or T7 exonuclease, the second exonuclease is full length RecE.

The kit further comprises a host cell expressing a second exonuclease, particularly, the host cell comprises a nucleic acid encoding of full length RecE, RecT, Redγ and RecA.

The kit further comprises one or more pre-prepared linear vectors.

Use of the aforesaid method or kit in construction of a targeting vector.

Use of the aforesaid method or kit in genotyping of mammalian cells.

Use of the aforesaid method or kit in DNA synthesis.

### BRIEF DESCRIPTION OF DRAWINGS

FIGS. 1A-1F show concerted action of in vitro assembly and full length RecE/RecT improves the efficiency of direct cloning. FIG. 1A: A schematic diagram of direct cloning of the 14-kb lux gene cluster from P. phosphoreum ANT-2200 genome. The p15A-cm vector and the target genomic DNA fragment have the same sequence at the very end. FIG. 1B: Longer homology arms increase the cloning efficiency of ExoCET. Linear vectors with 25-bp, 40-bp or 80-bp homology boxes were mixed with genomic DNA, respectively, and the reaction was carried out for 20 minutes at 25° C. with 0.02 U μL⁻¹ of T4pol before annealing and conversion to arabinose-induced E. coli GB05-dir. Error bars, s.d.; n=3. FIG. 1C: Optimizing the concentration of T4pol. The linear vector with 80-bp homology boxes and the genomic DNA was treated in the same manner as in (b), except for the different concentration of T4pol. FIG. 1D: Effect of incubation time of T4pol on cloning efficiency. As with (c), 0.02 U μL⁻¹ of T4pol was used, but with different incubation time. FIG. 1E: Higher copy number of ETgA increases ExoCET cloning efficiency. As with (d), the incubation time is 1 h, and then the in vitro assembled products were separately electroporated into arabinose-induced E. coli GB05-dir (with a copy of ETgA on the chromosome), containing GB2005 (with −5 copies of ETgA on pSC101 plasmid) of pSC101-BAD-ETgA-tet or containing GB05-dir (with −6 copies of ETgA) of pSC101-BAD-ETgA-tet. FIG. 1F: ExoCET increases the efficiency of direct cloning. As with (e), GB05-dir (ExoCET) containing pSC101-BAD-ETgA-tet was used, or T4pol treatment (ETgA) was not used, or arabinose induction (T4pol) was not used.

FIGS. 2A-2F show effect of different exonucleases on direct cloning of the lux gene cluster. The p15A-cm vector and the P. phosphoreum genomic DNA were treated with exonuclease, annealed and then electroporated into arabinose-induced E. coli GB05-dir. FIG. 2A: Initial detection of different exonucleases. FIGS. 2B-2D: Optimizing the concentrations of Kle, T5exo and T7exo. FIG. 2E: Comparison of cloning efficiency after 20 min digestion with T4pol, Kle, T5exo and T7exo at optimal concentrations. FIG. 2F: Optimizing the digestion temperature and time of T4pol (0.02 U μL⁻¹). Error bars, s.d.; n=3.

FIGS. 3A-3B show effect of annealing rate on direct cloning of the lux gene clusters. Error bars, s.d.; n=3. The p15A-cm vector and the P. phosphoreum genomic DNA were digested with 0.02 U μL⁻¹ T4pol and then annealed in different methods (A, B, C) and finally electroporated into arabinose-induced E. coli GB05-dir.

FIGS. 4A-4B is a flowchart of preparation of linear cloning vector and ExoCET direct cloning technique. FIG. 4A: The p15A-cm vector is prepared by PCR amplification and the primers employed carry a homology box of 80 nucleotides. FIG. 4B: The standard strategy for ExoCET direct cloning. The in vitro assembled product is transformed into arabinose-induced GB05-dir containing pSC101-BAD-ETgA-tet, and the correct recombinant is obtained by antibiotic screening.

FIGS. 5A-5D: The position of the 80-bp homology boxes between the p15A-cm and the 14-kb lux genomic DNA fragments: (FIG. 5A) Both homology boxes are at the very end; (FIGS. 5B, 5C) one is located 1 kb from the end and the other is at the very end; (FIG. 5D) Both were located 1-kb from the end. The reaction conditions are the same as in FIG. 1F. FIG. 5E: The number of colonies was obtained by ETgA, T4pol or ExoCET combining with the above four homology boxes. FIG. 5F: Using ExoCET and terminal homology boxes in GB2005, the direct cloning efficiency of 14-kb lux gene cluster was obtained by the different combinations of recombinant proteins expressed by pSC101 plasmid: ETg-no express RecA; Eg-no express RecA and RecT; Tg-no express RecA and RecE; pSC101-tet-empty vector. Error bars, s.d.; n=3.

FIGS. 6A-6C show direct cloning of the 106-kb salinomycin gene cluster using the Streptomyces albus genomic DNA digested with EcoRV or Cas9. FIG. 6A: Under the action of ExoCET, the salinomycin gene cluster was cloned from genomic DNA digested with EcoRV or Cas9 gRNA2/Cas9-gRNA7 and was then inserted into the pBeloBAC11 vector. The homology box (blue) was first inserted into the BAC vector and the BAC vector was linearized by BamHI digestion to be a direct cloning vector. The length of the homology box was marked at the end of the genomic DNA fragment. FIGS. 6B-6C: PvuII restriction analysis of recombinant DNA. The correct clones are marked by arrows.

FIGS. 7A-7D show comparison of the efficiency of ExoCET and Gibson assembly. FIG. 7A: A schematic diagram of direct cloning of a 45-kb DNA fragment containing the Wnt4 gene from the mouse genome using the terminal homology boxes. FIG. 7B: Number of colonies obtained by the ExoCET and Gibson methods. The mouse genomic DNA digested with p15A-cm and SwaI were treated with ExoCET and Gibson, respectively, and then transformed into the GB05 containing pSC101-BAD-ETgA-tet-dir, whether arabinose-induction (ExoCET or Gibson+ETgA) or non-induction (T4pol or Gibson). FIG. 7C: A schematic diagram of assembling multiple DNA fragments into plasmids by T4pol, ExoCET, Gibson and Gibson+ETgA. The size range of the DNA fragment is 1.0-kb to 5.4-kb, the size range of the assembled p5A plasmid is 29.8-kb to 54.9-kb and the plasmids are resistant to chloramphenicol (cm). FIG. 7D: The number of clones and the correct rate obtained by the experiment of multiple fragment assembly. The in vitro assembled product is transformed into GB05-dir containing pSC101-BAD-ETgA-tet, whether arabinose-induction (ExoCET or Gibson+ETgA) or non-induction (T4pol or Gibson).

FIGS. 8A-8D show construction of HIT (haplotype isogenic targeting) vectors for DPY30 using mammalian genomic DNA. FIG. 8A: A schematic diagram of the cloning of DPY30 stop codon using human genomic DNA digested with SpeI. After the direct cloning was completed, the C-terminus of DPY30 was labeled by the mVenus element of the Redαβ recombineering. FIG. 8B: Employing the genomic DNA isolated from human blood as a template, the recombinant DNA directly cloned by ExoCET was subjected to EcoRI digestion analysis. FIG. 8C: EcoRI digestion analysis of the recombinant DNA obtained by direct cloning through ExoCET and genomic DNA isolated from human embryonic kidney 293T cells. FIG. 8D: The PvuII digestion analysis of recombinant DNA obtained after inserting the mVenus element by Redαβ recombineering. All clones obtained are correct, with lane 11 being the control. The correct clone is marked by arrows.

FIGS. 9A-9C show construction of HIT (haplotype isogenic targeting) vectors for Dpy30 using mouse genomic DNA. FIG. 9A: A schematic diagram of the cloning of Dpy30 stop codon using mouse genomic DNA digested with BamHI+KpnI. Once directly cloned, the C-terminus of DPY30 was tagged with a mVenus cassette using Redαβ recombineering. FIG. 9B: EcoRI digestion analysis for the recombinant DNA obtained by direct cloning through ExoCET and genomic DNA isolated from mouse melanoma B16 cells. FIG. 9C: NheI digestion analysis of the recombinant DNA obtained by inserting the mVenus element of the Redαβ recombinant engineering. All clones obtained were correct, with lane 11 being the control. The correct clone is marked by arrows.

FIGS. 10A-10B show genotyping of mammalian cells using ExoCET. FIG. 10A: A schematic diagram of genotyping using ExoCET. Restriction sites are located upstream and downstream of the targeting element, respectively. FIG. 10B: Genotyping of mouse embryonic stem cells targeted by Kmt2d-AID-neo with kanamycin resistance using ExoCET. A DNA fragment containing a targeting element was released from the genome using SspI and SpeI. ExoCET cloning is performed using 10 μg of restriction-digested genomic DNA and PCR-amplified p15A-cm vector. Targeting fragments and wild-type fragments cloned into the p15A vector can be separated by double streak and restriction enzyme digestion.

FIG. 11 shows EcoRV+PstI restriction analysis of chloramphenicol resistant colonies obtained by ExoCET genotyping of Klf4-Venus-neo-targeted mouse embryonic stem cells.

### DETAILED DESCRIPTION

DNA recombination engineering is a genetic engineering technique for modifying DNA molecules in E. coli cells, which is mediated by homologous recombination of the phage syn/exo proteins (mainly Redα and Redβ). DNA recombination engineering was first discovered in the E. coli sbcA (recBC repressor) strain, which has an activity that efficiently mediates homologous recombination between DNA molecules with homology boxes. The sbcA strain was discovered in a classic experiment by A J Clark looking for a homologous recombination pathway in E. coli. He used the recBC strain, which is very sensitive to DNA damage, to screen for its inhibitor, and found sbcA mutant strains with RecE and RecT expression activities. Subsequent studies have shown RecE and RecT are expressed by Rac phage integrated on chromosomes, which function identically to phage Redα and Redβ, and only 280 amino acids at the C-terminus of RecE protein are expressed in the sbcA mutant strain. The truncated RecE is similar to Redα (266 amino acids) and is a 5′ to 3′ exonuclease. RecT is similar to Redβ and is a single-strand annealing protein (SSAP).

RecE/RecT and Redα/Redβ belong to the 5′to 3′ exonuclease/SSAP syn/exo protein pairs, and a specific protein-protein interaction potential between each pair of proteins is necessary for homologous recombination of double-stranded DNA. Redα/Redβ-mediated homologous recombination occurs mainly on the replication fork and requires simultaneous replication. Although recombinant engineering techniques through truncated RecE/RecT was initially discovered, Redα/Redβ was used to modify DNA molecules because the latter were more efficient. The characteristics of RecE/RecT is studied, and it is found that the 600 amino acid residues at the N-terminus of RecE changes their recombination activity from replication-dependent to replication-independent. Therefore, two linear DNA molecules can form a circular plasmid by efficient homologous recombination through a very short homologous box. Compared with the Redα/Redβ recombination engineering, this linear-linear recombination mechanism has different applications, such as directly cloning large DNA fragments from genome or performing multiple DNA fragment assembly.

The disclosure provides a method of homologous recombination (linear-linear recombination) between two or more target linear nucleic acid molecules sharing at least one homologous sequence. The method comprises the mixture of target linear nucleic acid molecules treated with a first exonuclease; then the treated target linear nucleic acid molecules is subjected to homologous recombination in the presence of a second exonuclease and an annealing protein. The second exonuclease can be RecE, and the amino acid sequence of full length RecE from E. coli K12 is disclosed in WO2011/154927. Or the second exonuclease may also be truncated RecE, and the truncated forms of RecE including RecE protein consisting of amino acids 588-866, 595-866, 602-866 or 606-866.

Homologous recombination is mediated by the second exonuclease and the annealing protein. In some embodiments, the annealing protein used in the methods is a related art. Particularly, the annealing protein is RecT or a fragment thereof (derived from Rac phage). More particularly, the annealing protein is the full length RecT and the second exonuclease is the full length RecE. However, any other suitable annealing protein can be used as long as the annealing protein interacts with the exonuclease used. Linear-linear recombination can occur in the host cells lacking RecT expression, such as E. coli strain GB2005, possibly due to the presence of certain endogenous RecT-like activities. However, the efficiency of linear-linear recombination mediated by full length RecE is significantly increased in the presence of RecT.

The methods of the disclosure can be affected in whole or in part in a host cell. Suitable host cells include cells of many species, including parasites, prokaryotes, and eukaryotes, but bacteria such as Gram-negative bacteria are preferred hosts. More particularly, the host cell is an enteric bacterial cell such as Salmonella, Klebsiella, Bacillus, Neisseria, Photorhabdus or Escherichia coli cells (the method of the disclosure plays an effective role in all E. coli strains that have been tested). A preferred host cell is E. coli K12. However, it should be noted that the methods of the disclosure are equally applicable to eukaryotic cells or organisms, such as fungal, yeast, plant or animal cells. This system has been shown to be functional in mouse's ES cells and it is reasonable to speculate that it is also functional in other eukaryotic cells. The host cell is typically an isolated host cell, but can be unisolated host cells.

The host cell of the disclosure comprises a nucleic acid encoding an exonuclease (particularly full length RecE), an annealing protein (particularly RecT) and Redγ. In some embodiments, the host cell further comprises a nucleic acid encoding RecA. Particularly, the host cell expresses RecE, RecT and Redγ, and optionally RecA. More particularly, the host cell expresses RecE, RecT, Redγ and RecA.

The exonuclease, annealing protein, Redγ and/or RecA of the disclosure can be a recombinant expression product from a foreign DNA in a host cell, for example, expressed by a vector transformed into a host cell. An example of a suitable vector is the pSC101 plasmid, although other suitable vectors can also be used. Any suitable promoter can be used to drive the expression of these proteins. However, in the case of expressing RecE, an inducible promoter such as an arabinose-inducible promoter (P_{BAD}) or a rhamnose-inducible promoter (P_{RhaSR}) is preferred. These promoters are well known in the art.

The host cell of the disclosure expresses an exonuclease, an annealing protein, Redγ and/or RecA by the inducible promoters on a plasmid vector or a chromosome. Particularly, the exonuclease, annealing protein, Redγ and/or RecA are expressed in the host cell by a plasmid vector. More particularly, the exonuclease, annealing protein, Redγ and/or RecA are simultaneously expressed in the host cell by the plasmid vector and the chromosome.

The genome of the E. coli K12 host cell consists of an endogenous copy of the full-length recE gene and the recT gene, which are present in the Rac phage that has been integrated into the host genome. However, since the gene is silent, the expression of full-length RecE cannot naturally occur from the integrated gene. Thus, in embodiments where the 5′ to 3′exonuclease is expressed by exogenous DNA, the method can be carried out in the absence of an endogenous recE gene.

Host cells transformed with the encoding as above nucleic acid molecule of the exonuclease are also provided. Particularly, the exonuclease is expressed by the nucleic acid molecule, and thus the disclosure also provides the host cell expressing the exonuclease enumerated in the method of the disclosure. The exonuclease is particularly expressed under the control of an inducible promoter, such as an arabinose-inducible promoter (P_{BAD}) or a rhamnose-inducible promoter (P_{RHaSR}).

In the foregoing embodiments, the methods of the disclosure may be affected in whole or in part in vitro. For example, purified 5′ to 3′ exonuclease and annealing protein (particularly purified RecE and RecT proteins) can be used, or an extract of E. coli cell expressing the 5′ to 3′ exonuclease and the annealing protein are used. When the method is carried out in vitro, it is advantageous to pretreat the first and second linear target nucleic acid molecules to expose single-stranded homologous ends.

Linear-linear recombination requires that at least one homologous sequence must be shared between the target linear nucleic acid molecules in which homologous recombination occurs. In some embodiments, the first target nucleic acid molecule shares a homologous sequence with the second target nucleic acid molecule to perform the linear-linear recombination between the first and second target nucleic acid molecules, to produce a linear product. In embodiments in which linear-linear recombination occurs between the first and second linear nucleic acids and one or more additional linear nucleic acids to form a linear product, each linear nucleic acid shares a homologous sequence with the linear nucleic acid that forms its neighbor in the linear products of the linear-linear recombination reaction. In embodiments in which linear recombination occurs between the first and second linear nucleic acids and one or more additional linear target nucleic acid molecules, to form a cyclic product, each linear nucleic acid shares a homologous sequence with linear nucleic acid that forms its neighbor in the cyclic product of the linear-linear recombination reaction. In some embodiments, the first target nucleic acid molecule and the second target nucleic acid molecule share two homologous sequences to perform a linear-linear recombination between the first and second target nucleic acid molecules, to form a cyclic molecule. Those skilled in the art know how to design homologous sequences to form linear or cyclic molecules.

Particularly, at least one homology box is at the very end of each linear fragment. When the homology boxes are at the very end of each linear fragment and the different homology boxes are at the other end, these homologous sequences or ‘homologous boxes’ produce the optimal configuration, and the construction of these homology boxes enables recombination to generate a ring. Linear recombination can occur when the homology box is not at the end, but the efficiency is reduced. Thus, in a preferred embodiment, at least one at least one homologous sequence is located at the outermost end of one or both ends of the target nucleic acid molecule. In some embodiments, the at least one homologous sequence is internal to the certain target nucleic acid molecule.

The homologous sequences of the disclosure are at least 4, at least 6, at least 10, at least 20, at least 30, at least 40, at least 50, at least 60, at least 70, at least 80, at least 90, at least 100 nucleotides in length. For example, in some embodiments, the homologous sequences are 4-6, 6-9, 6-30, 6-100, 10-20, 20-29, 20-40, 20-50, 10-100, 25-30, 25-40, 25-50, 30-40, 30-50, 40-50, 40-80 or more than 80 nucleotides. The efficiency of homologous recombination increases with the length of the homology boxes used, so longer homology boxes can be used.

‘Homologous’ between two nucleic acid molecules means that when the sequences of two nucleic acid molecules are aligned, there are many nucleotide residues that are identical at the same position in the sequence. The degree of homology is easy to calculate.

In some embodiments, the methods of the disclosure comprise joining together a plurality of linear nucleic acid molecules to form a circular nucleic acid molecule, such as a circular plasmid. Each target nucleic acid molecule shares a at least one homologous sequence with a target nucleic acid molecule that forms its neighbor in the resulting cyclic product and is subjected to linear-linear recombination in accordance with the methods of the disclosure. The number of target nucleic acid molecules is 2, 3, 4, 5, 6, 7, 8, 9, 10, 11, 12, 13, 14, 15, 16, 17, 18, 19, 20 or more.

In some embodiments, at least one of the target linear nucleic acid molecules comprises a selection marker to allow selection of the correct recombinant. Any suitable selection marker can be used in the disclosure. In some embodiments, the selection marker is an antibiotic resistance gene, e.g., a resistance gene for chloramphenicol, ampicillin, kanamycin, or blasticidin.

The target linear nucleic acid molecule can be derived from any suitable source. For example, nucleic acid sequences from eukaryotes or prokaryotes can be included. In some embodiments, the first target linear nucleic acid molecule is genomic DNA. Typically, the genomic DNA is a genomic DNA fragment. The genomic DNA particularly consists of a target sequence. In some embodiments, a genomic DNA fragment can be obtained by cleavage or digestion of genomic DNA (for example, using a restriction enzyme) to obtain a complete target sequence containing. In some embodiments, the first target linear nucleic acid molecule (such as, a genomic DNA fragment, a cDNA library member, or a BAC-derived fragment) comprises a target sequence of 2-kb or longer (e.g., 2.5-kb or longer, 4-kb or longer, 5-kb or longer, 7.5-kb or longer, 10-kb or longer, 15-kb or longer, 20-kb or longer, 25-kb or longer, 40-kb or longer, 50-kb or longer, 75-kb or longer or −100 kb or longer). Particularly, the target sequence is the entire region between the homology boxes at either end of the first target linear nucleic acid molecule. For example, a gene cluster encoding a secondary metabolite pathway or a fatty acid synthesis pathway. In some embodiments, the methods of the disclosure can be used to directly clone a DNA region from a human or non-human animal genome. For example, regenerative therapies for health research or for correction by gene targeting. For example, in some embodiments, the first target nucleic acid molecule comprises or consists of a genomic DNA fragment from a human or non-human animal. The genomic DNA fragment can comprise a target sequence, such as a gene comprising a mutation, wherein the mutation results in a disease or condition and the modification of the mutation to a wild type sequence can treat or prevent the disease or condition. In embodiments where the first target nucleic acid molecule is a genomic DNA fragment, the second target nucleic acid molecule is particularly a linear cloning vector.

In embodiments where the first target nucleic acid molecule is a genomic DNA fragment, The method comprises generating a first target nucleic acid molecule by digesting or cleaving genomic DNA to obtain a linear genomic DNA fragment comprising the target sequence, then, the first exonuclease is used to treat the mixture of the genomic DNA fragment and the linear cloning vector, processing the steps of cleaving the target nucleic acid molecule and annealing to join the target nucleic acid molecule, and then the mixture of the treated nucleic acid molecules is transferred into host cells. The second target nucleic acid molecule particularly comprises a selection marker.

In one embodiment, the methods of the disclosure comprise the step of joining of DNA molecules in vitro.

The joining process in vitro comprises exonuclease digestion followed by annealing.

The exonuclease is T4 polymerase.

The joining process in vitro comprises Gibson assembly.

The joining process in vitro comprises DNA synthesis by DNA polymerase with or without exonuclease followed by annealing.

The joining process in vitro comprises annealing by a single-stranded annealing protein, such as RecA/RAD51, Redβ, RecT. Pluβ or RAD52.

Host cells used for homologous recombination are E. coli cells.

Host cells for homologous recombination are E. coli cell expressing full length RecE and/or RecT.

Host cells for homologous recombination are E. coli cell expressing full length RecE, RecT and/or Redγ.

Host cells for homologous recombination are E. coli cell expressing truncated RecE, RecT and/or Redγ.

Host cells for homologous recombination are any bacterial host cell expressing full length RecE and/or RecT.

Host cells for homologous recombination are E. coli cell expressing Redα, Redβ and/or Redγ.

The host cell for homologous recombination is Saccharomyces cerevisiae cells.

Kits for use in the disclosure are provided. In some embodiments, the kits comprise a nucleic acid encoding an exonuclease as described herein. In some embodiments, the kit comprises an exonuclease as described herein. Particularly, the first exonuclease is T4 DNA polymerase (T4pol), Klenow fragment of DNA Polymerase I (Kle), T7 DNA polymerase (T7pol), Exonuclease III (ExoIII), Phusion DNA polymerase (Phu), T5 exonuclease (T5exo), T7 exonuclease (T7exo) and Lambda exonuclease (λexo), and the second exonuclease is full length RecE. More particularly, the kits comprise a host cell as described herein. For example, in some embodiments, the host cells in a kit comprises a nucleic acid encoding a full length RecE, RecT, Redγ, and RecA described herein under the control of an inducible promoter. The kits may also include one or more pre-prepared linear cloning vectors.

Another preferred application of the disclosure relates to the assembly of linear nucleic acid molecules in synthetic biology, particularly linear DNA. Thus, in some embodiments, the first and second target nucleic acid molecules are linear, and the method further comprises contacting the first and second target nucleic acid molecules with one or more other linear target nucleic acid molecules (For example, 1, 2, 3, 4, 5, 6, 7, 8, 9, at least 10, at least 20 other target nucleic acid molecules) in the presence of a 5′ to 3′ exonuclease and an annealing protein, to produce a linear or circular product. In a preferred embodiment, homologous recombination between the first and second target nucleic acid molecules and one or more other target nucleic acid molecules results in the production of genes, operons, chromosomes or whole genomes. Synthetic biological assembly of DNA nucleic acids has been used to generate genes, operons, chromosomes, or recently used to generate whole genomes. In an embodiment of the disclosure, the combination of the first exonuclease and the second exonuclease significantly increases the assembly efficiency of the linear nucleic acid molecule, the disclosure will be a preferred method for the assembly of synthetic biological DNA in commerce and research.

Another preferred application of the disclosure is to construct a haplotype isogenic targeting vector, which can directly clone a 5 to 10-kb DNA fragment from mammalian genome using the method of the disclosure as an isogenic homology box, and these DNA fragments are the identical genes and maintains a polymorphic haplotype, which is called a haplotype isogenic targeting vector, that is the so called haplotype isogenic targeting (HIT) vector. The selection marker and other functional elements are then inserted into the HIT vector by recombinant engineering to obtain a vector for targeting. Another preferred application of the disclosure is the genotyping of mammalian cells. The DNA fragment containing the complete targeting element is cloned from the genome of the possible target embryonic stem cells by the method of the disclosure, and the recombinant plasmid obtained by the cloning is subjected to restriction analysis and DNA sequencing, and the cell is successfully determined according to the result.

### EXAMPLES

### Materials and Method

### Strains and Plasmids

E. coli GB2005 was derived from DH10B by deleting fhuA, ybcC and recET. GB05-dir was derived from GB2005 by integrating the P_{BAD}-ETgA operon (full length recE, recT, redγ and recA under the arabinose-inducible P_{BAD} promoter) at the ybcC locus. GB08-red was derived from GB2005 by integrating the P_{BAD}-gbaA operon (redγ, redβ, redα and recA under the arabinose-inducible P_{BAD} promoter) at the ybcC locus.

pSC101-BAD-ETgA-tet conveys tetracycline resistance and carries the P_{BAD}-full length ETgA operon and a temperature sensitive pSC101 replication origin which replicates at 30° C. but not at 37° C. so it can be easily eliminated from the host by temperature shift in the absence of selection.

### Preparation and Digestion of Genomic DNA

Gram-negative Photobacterium phosphoreum ANT-2200 and Photorhabdus luminescens DSM15139 were cultured overnight in 50 mL of medium. After centrifugation the cells were resuspended thoroughly in 8 mL of 10 mM Tris-Cl (pH 8.0). Five hundred microliters of 20 mg mL⁻¹ proteinase K and 1 mL of 10% SDS were added and incubated at 50° C. for 2 h until the solution became clear. Genomic DNA was recovered from the lysate by phenol-chloroform-isoamyl alcohol (25:24:1, pH 8.0) extraction and ethanol precipitation. The DNA was dissolved in 10 mM Tris-Cl (pH 8.0) and digested with BamHI+KpnI for cloning of the 14-kb lux gene cluster.

Gram-positive Streptomyces albus DSM41398 was cultured in 50 mL of tryptic soy broth at 30° C. for 2 days. The genomic DNA was isolated. After centrifugation the cells were resuspended thoroughly in 8 mL of SET buffer (75 mM NaCl, 25 mM EDTA, 20 mM Tris, pH 8.0) and 10 mg lysozyme was added. After incubation at 37° C. for 1 h, 500 μL of 20 mg mL⁻¹ proteinase K and 1 mL of 10% SDS were added and incubated at 50° C. for 2 h until the solution became clear. Three and a half milliliters of 5 M NaCl was added into the lysate. Genomic DNA was recovered from the lysate by phenol-chloroform-isoamyl alcohol (25:24:1, pH 8.0) extraction and ethanol precipitation. The DNA was dissolved in 10 mM Tris-Cl (pH 8.0).

Genomic DNA was purified from mouse melanoma B16 cells, human embryonic kidney 293T cells and human blood using Qiagen Blood & Cell Culture DNA Kits according to the manufacturer's instructions, except DNA was recovered from the Proteinase K treated lysate by phenol-chloroform-isoamyl alcohol (25:24:1, pH 8.0) extraction and ethanol precipitation. The DNA was dissolved in 10 mM Tris-Cl (pH 8.0). Restriction digested genomic DNA was extracted with phenol-chloroform-isoamyl alcohol (25:24:1, pH 8.0) and precipitated with ethanol. The DNA was dissolved in 10 mM Tris-Cl (pH 8.0). End cut pipette tips were used to avoid shearing genomic DNA.

The genomic DNA of P. luminescens DSM15139 was digested with XbaI for plu3535-plu3532 cloning, and XbaI+XmaI for plu2670 cloning. The genomic DNA of S. albus was digested with EcoRV or Cas9-gRNA complexes for cloning of the salinomycin gene cluster. The mouse genomic DNA was digested with HpaI for Prkar1a cloning, BamHI+KpnI for Dpy30 cloning, and SwaI for Wnt4 or Lmbr1l-Tubala cloning. The human genomic DNA was digested with SpeI for DPY30 cloning, NdeI+BstZ17I for IGFLR1-LIN37 cloning, BstZ17I for IGFLR1-ARHGAP33 cloning and NdeI for ZBTB32-LIN37 cloning. Digested genomic DNA was extracted with phenol-chloroform-isoamyl alcohol (25:24:1, pH 8.0) and precipitated with ethanol. The DNA was dissolved in ddH₂O and concentrated to 1 μg μl⁻¹. End cut pipette tips were used to avoid shearing genomic DNA. Ten micrograms of digested genomic DNA were used for ExoCET cloning.

### Cas9 Digestion of S. Albus Genomic DNA

S. pyogenes Cas9 protein was purchased from New England Biolab. Cas9 digestion of S. albus genomic DNA was carried out in an 800 μL reaction system containing 80 μL of 10×Cas9 reaction buffer (NEB), 80 μg of genomic DNA, 40 μg of gRNA-2, 40 μg of gRNA-7 and 20 μg of Cas9. Since the cleavage efficiency of Cas9 was severely affected by the purity of the DNA substrate, in this experiment, the S. albus genomic DNA needed to be extracted three times with phenol-chloroform-isoamyl alcohol (25:24:1, pH 8.0) to ensure the cleavage efficiency of Cas9. After the resulting product was incubated at 37° C. for 6 h, 100 μg of RNase A (Thermo Scientific) was added, and after incubation at 37° C. for 1 h, 100 μg of proteinase K (Roche) was then added, and incubation was continued at 50° C. for 1 h. The genomic DNA was then extracted once with phenol-chloroform-isoamyl alcohol (25:24:1, pH 8.0), and after ethanol precipitation, it was dissolved in an appropriate amount of ddH₂O to a final concentration of about 1 μg uL⁻¹. Finally, 10 μg of genomic DNA cleaved by Cas9 protein was used for the cloning experiment of the method of the disclosure.

### Preparation of Linear Cloning Vector

Using p15A-Pamp-luxABECD plasmid (Genebridge) as a template, the p15A-cm vector was PCR-amplified with PrimeSTAR Max DNA Polymerase (Takara), the primers used (Table 1) consist of 80 nucleotide homology boxes and were purified by PAGE. The PCR product eliminated the interference of the primers on subsequent experiments by gel recovery. The kit used was QIAquick gel extraction kit (Qiagen). Finally, the DNA was eluted with ddH₂O at a concentration of approximately 200 ng/μL, and 200 ng was used for the ExoCET cloning experiment.

The pBeloBAC11 vector used to clone the salinomycin gene cluster and the pBAC2015 vector used to clone plu3535-3532 were constructed. BAC vectors were linearized with BamHI to expose both homology arms, and extracted with phenol-chloroform-isoamyl alcohol (25:24:1, pH 8.0) and precipitated with isopropanol. The DNA was dissolved in ddH2O and concentrated to 1 μg/μ. One microgram of linear BAC vectors were used for ExoCET cloning.

TABLE 1
Oligonucleotides amplified by p15A-cm linear vector

| Gene | Primer | Sequences (5′-3′) |
|---|---|---|
| plu2670 | plu2670-1 | ctgggaaaactacggtgagcatatgtttaccacttatcagagaaccgtgaatattgcgttaaaccg |
| | | atgaactgcccgggTTACGCCCCGCCCTGCCACTC (SEQ. ID. NO. 1) |
| | plu2670-2 | aaatccatgattaccaacccactcccaaagatattataaaatctcaacatgctgatttgatcctttgga |
| | | atgggctaaatctagaGGTAACGAATCAGACAATTGACG( SEQ. ID. NO. 2) |
| Mouse | mPrkar1a-1 | agatgacgccctctccactctgcataccaattccacataaccactcttcaaattataatgctcacaga |
| Prkar1a | | ccctctaaggttTTACGCCCCGCCCTGCCACTC (SEQ. ID. NO. 3) |
| | mPrkar1a-2 | catagtgacccattgttatagagtacttagctatgcctcagagtaatgctcttgatctaggtgctttctt |
| | | tttacttgttGGTAACGAATCAGACAATTGACG (SEQ. ID. NO. 4) |
| Mouse | mDpy30-1 | ggattttataagtgaagggtgacatttgtagtattccttaccccgtctggttcctcatacgcattaactc |
| Dpy30 | | ataatccttggtaccTTACGCCCCGCCCTGCCACTC (SEQ. ID. NO. 5) |
| | mDpy30-2 | tgccattcagaagacctaggtttgattcctagcacccactgatgctaacaaccagacataatgcca |
| | | gttctaggggatccGGTAACGAATCAGACAATTGACG (SEQ. ID. NO. 6) |
| Mouse | mWnt4-1 | gtttaactccataacacacacacacacacatgcacacgcacatgcacacgcgcactcacaccac |
| Wnt4 | | aaaatcacacaatttTTACGCCCCGCCCTGCCACTC (SEQ. ID. NO. 7) |
| | mWnt4-2 | ctgtgctcagagattgagtgccctacatataacaggacacaaactcttccttactttctgattgcca |
| | | catggtccatttGGTAACGAATCAGACAATTGACG (SEQ. ID. NO. 8) |
| Mouse | mLT-1 | ctatgccataggaatttggaggaaattaggatactggtccattaattatttccaacggatgcagtca |
| Lmbr1l- | | gaatgtatatttTTACGCCCCGCCCTGCCACTC (SEQ. ID. NO. 9) |
| Tubala | mLT-2 | ttagagcagacagtgctataaccgttgagccatctctccagtecctggacacactgattttaagttg |
| | | acattaaaatttGGTAACGAATCAGACAATTGACG (SEQ. ID. NO. 10) |
| human | hDpy30-1 | ccaccacgccaggctaattttagataataccctattacctctcaggtgtgtctaaatttttctttgatg |
| DPY30 | | gaaaaactagtTTACGCCCCGCCCTGCCACTC (SEQ. ID. NO. 11) |
| | hDpy30-2 | agttatgtcagtttgctattataatgtttattattattecttgaataaaaaagaatatgcacctaataaac |
| | | gttgactagtGGTAACGAATCAGACAATTGACG( SEQ. ID. NO. 12) |
| human | hIL-1 | Agtggccctagagggtcggtaaggagccaagggcctatgcaaggatgctcacacacctcccat |
| IGFLR1- | | ccccaccttcccagacacatatgTTACGCCCCGCCCTGCCACTC( SEQ. ID. NO. 13) |
| LIN37 | hIL-2 | gtgctgtccaagcttggctcatctggggtttgctgggcttaacacccaataaagaactttgctgact |
| | | actaagcccagtaGGTAACGAATCAGACAATTGACG (SEQ. ID. NO. 14) |
| human | hIA-1 | acggccggcgactgctcctcagtgaggaggcgtcactcaatatccctgcagtggcggccgccc |
| IGFLR1- | | atgtgatcaaacggtaTTACGCCCCGCCCTGCCACTC (SEQ. ID. NO. 15) |
| ARHGA | hIA-2 | gtgctgtccaagcttggctcatctggggtttgctgggcttaacacccaataaagaactttgctgact |
| P33 | | actaagcccagtaGGTAACGAATCAGACAATTGACG (SEQ. ID. NO. 16) |
| human | hZL-1 | cctagagggtcggtaaggagccaagggcctatgcaaggatgctcacacacctcccatccccac |
| ZBTB32- | | cttcccagacacatatgTTACGCCCCGCCCTGCCACTC (SEQ. ID. NO. 17) |
| LIN37 | hZL-2 | aagaactagactagaagttggaaaacaggtttcactgcatcccttcacagtgaatggcccttctctt |
| | | cagcactcatatgGGTAACGAATCAGACAATTGACG (SEQ. ID. NO. 18) |
| | mVenus- | tcctagcatcttatcttttaaaaaacaaggcgcagtttgaagatcgaaatGAAGTGCATAC |
| | mDpy30-1 | CAATCAGGAC (SEQ. ID. NO. 19) |
| | mVenus- | atcatgtaaatctacagtagcaaccaaatgtttcttctatcccaagaaaCTATTTAGTCAA |
| | mDpy30-2 | GCGCTCTTGTCGTCGTCATCCTTGTAG (SEQ. ID. NO. 20) |
| | mVenus- | ttctagcatcttatcttttaaaaaacaaggcacagtttgaagatcgaaacGAAGTGCATAC |
| | hDPY30-1 | CAATCAGGAC (SEQ. ID. NO. 21) |
| | mVenus- | atcatgtaaatctacagtagcaactaaatttttctgttcttcccattaagCTATTTAGTCAA |
| | hDPY30-2 | GCGCTCTTGTCGTCGTCATCCTTGTAG (SEQ. ID. NO. 22) |

Lower-case letters in Table 1 represent the homologous box sequence.

### Preparation of PCR Products for Multiple Fragment Assembly

The DNA fragments with 40 bp homologies used in the multi-piece assembly experiment were PCR amplified using genomic DNA of P. luminescens DSM15139 as the template and PrimeSTAR Max DNA Polymerase (Takara) according to the manufacturer's instructions. The PCR products were extracted from agarose gels after electrophoresis and purified using the QIAquick gel extraction kit (Qiagen) according to the manufacturer's instructions, except that DNA was eluted from the column with ddH₂O and concentrated to 200 ng μL⁻¹. 250 ng of each fragment was used for DNA assembly.

### Preparation of mVenus-PGK-Neo DNA Cassette

The mVenus-PGK-neo cassette was amplified from pR6K-2Ty1-2PreS-mVenus-Biotin-PGK-em7-neo with PCR using the proof reading PrimeSTAR Max DNA Polymerase (Takara) according to the manufacturer's instructions. The primers are listed in Table 1. The PCR products were purified with QIAquick PCR Purification Kit (Qiagen) according to the manufacturer's instructions, except that DNA was eluted from the column with ddH2O and concentrated to 100 ng μL⁻¹. Two hundred nanograms of the cassette was used for recombineering.

### In Vitro Assembly

Ten micrograms of genomic DNA and 200 ng of 2.2-kb p15A-cm linear vector (1 μg of 8-kb linear BAC vector) were assembled in 20 μL reactions consisting of 2 μL of 10×NEBuffer 2.1 and 0.13 μL of 3 U μL⁻¹ T4pol (NEB, cat. no. M0203). Assembly reactions were prepared in 0.2 ml PCR tubes and cycled in a thermocycler as follows: 25° C. for 1 h, 75° C. for 20 min, 50° C. for 30 min, then held at 4° C. For multi-piece assembly, 250 ng of each fragment was added and a chew-back time of 20 min was used. The in vitro assembly products were desalted at room temperature for 30 min by drop dialysis against ddH₂O using Millipore Membrane Filters (Merck-Millipore, cat. no. VSWP01300) prior to electroporation. All experiments were performed in triplicates.

Assembly reactions with other exonucleases were cycled as follows: T5exo: 50° C. for 30 min, then held at 4° C.; T7exo: 25° C. for 20 min, 50° C. for 30 min, then held at 4° C.; Kle, T7pol and λexo: 25° C. for 20 min, 75° C. for 20 min, 50° C. for 30 min, then held at 4° C.; ExoIII: 37° C. for 20 min, 75° C. for 20 min, 50° C. for 30 min, then held at 4° C.; Phu: 37° C. for 20 min, 50° C. for 30 min, then held at 4° C. Gibson assembly was performed at 50° C. for 30 min with Gibson Assembly Master Mix (NEB, cat. E2611), then held at 4° C.

### Preparation of Electrocompetent E. Coli Cells

E. coli GB05-dir containing plasmid pSC101-BAD-ETgA-tet was cultured (OD600=3-4) overnight at 30° C. in LB supplemented with 4 μg/mL tetracycline. 40 μL of overnight culture (OD600=3-4) was transferred to 1.4 mL LB supplemented with appropriate antibiotics, then the mixture was placed on an Eppendorf thermomixer at 30° C. and incubated at 950 rpm for 2 h (OD600=0.35-0.4). 35 μL of 10% L-arabinose (w/v, in ddH₂O) was added to induce expression of recombinant enzyme (ETgA or gbaA), and the incubation was continued for 40 min at 37° C. (OD600=0.7 to 0.8). The cells were collected by centrifugation at 9,400 g for 30 sec at 2° C.

The supernatant was discarded and the cell pellet was suspended in 1 mL of ice-cold ddH₂O. The cells were collected by centrifugation at 9,400 g for 30 sec at 2° C. The supernatant was discarded and the cell pellet was suspended in 1 mL of ice-cold ddH₂O. The cells were repeatedly centrifuged, resuspended, and centrifuged again, and the cells were suspended with 20 μL of ice ddH₂O. Then 5 μL of desalted in vitro assembled product was added, while in the mVenus-PGK-neo element insertion experiment, the mixture of 200 ng of plasmid and 200 ng of PCR product was added. The mixture of cells and DNA was transferred to a 1-mm cuvettes and electroporated with an Eppendorf electroporator 2510 at a voltage of 1350 V, a capacitance of 10 μF, and a resistance of 600Ω. 1 mL LB was added to the cuvette, washed the cells and transferred it to a 1.5 mL tube with holes, and then placed on the Eppendorf thermomixer at 950 rpm for 1 h at 37° C. Finally, an appropriate amount of the bacterial solution was spread to an LB plate supplemented with a suitable antibiotic (15 μg/mL chloramphenicol or 15 μg/mL kanamycin) and incubated at 37° C. overnight.

### Example 1

### Concerted Action of In Vitro Assembly and Full Length RecE/RecT Improves the Efficiency of Direct Cloning

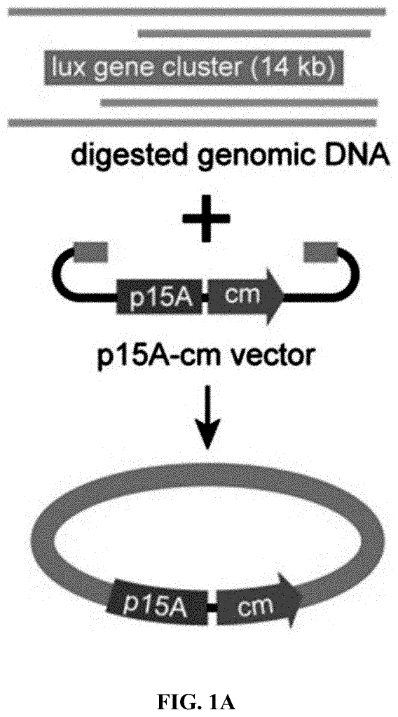

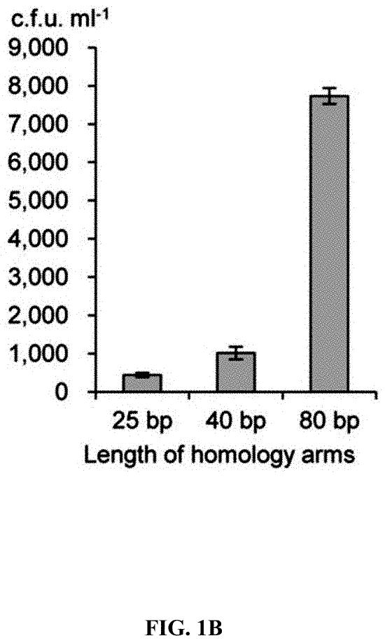

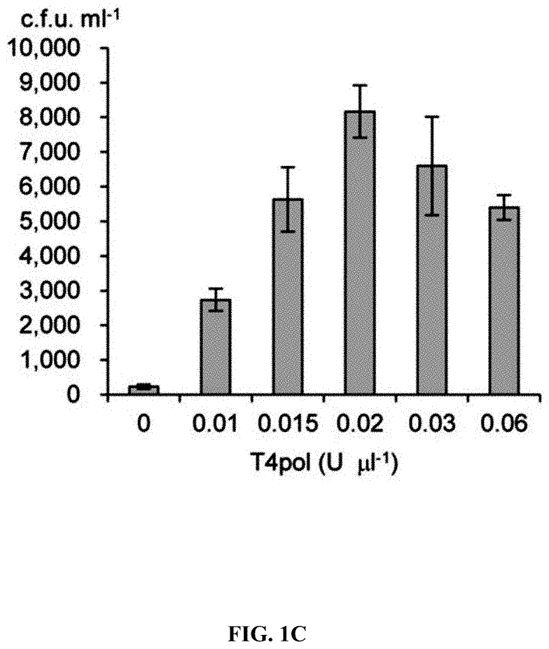

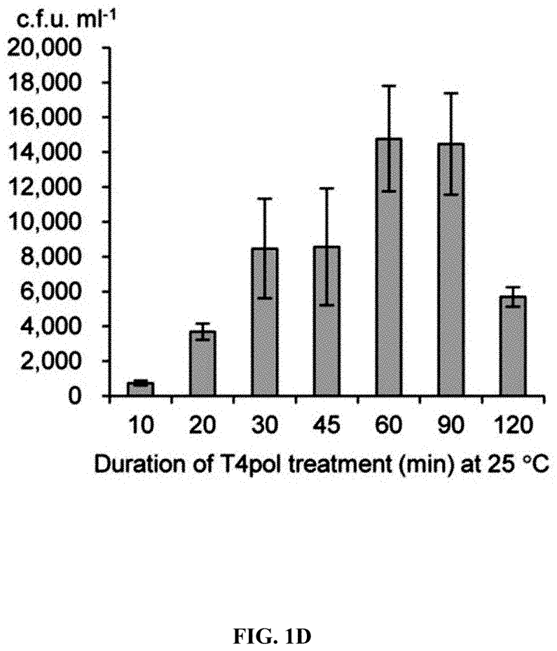

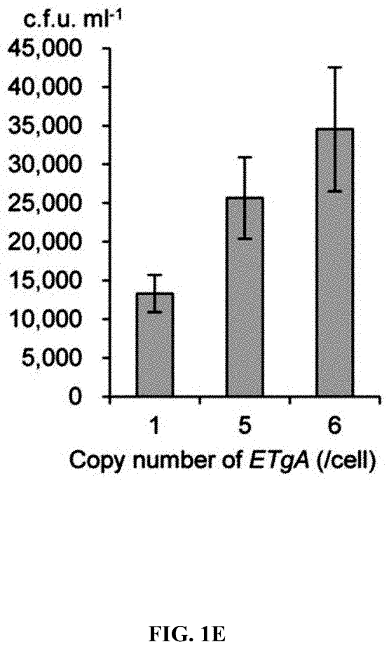

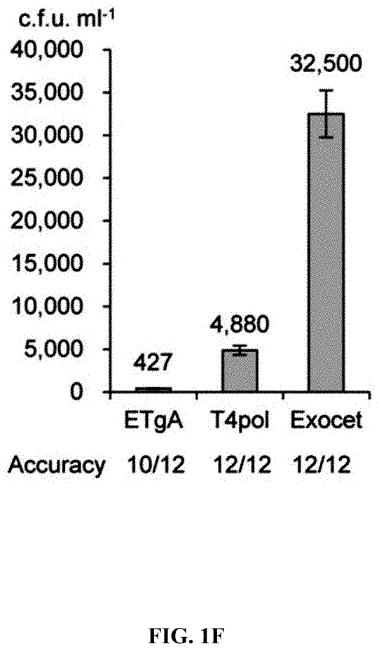

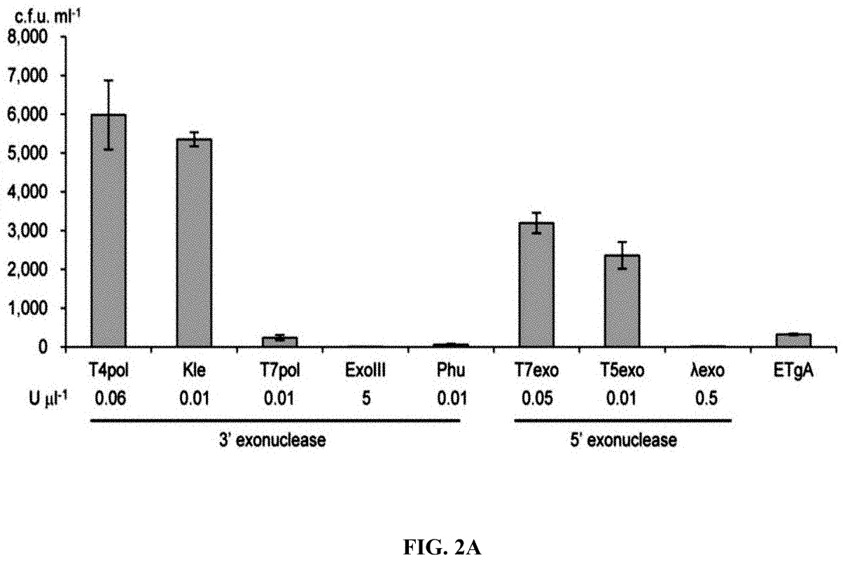

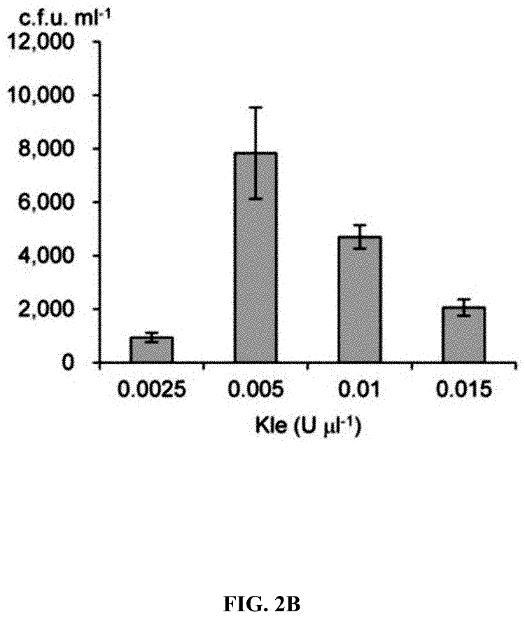

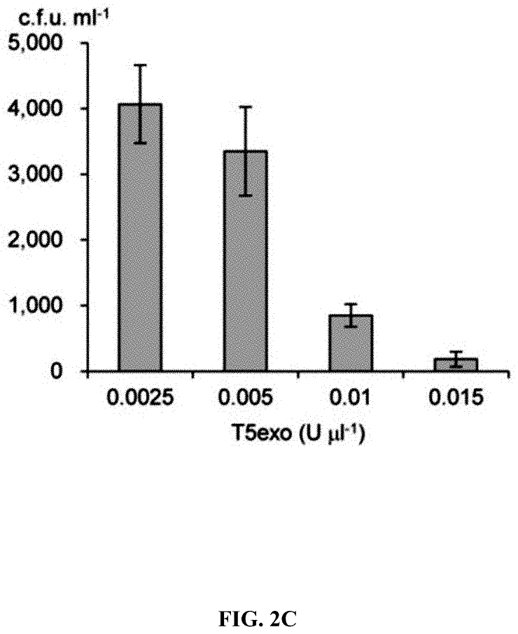

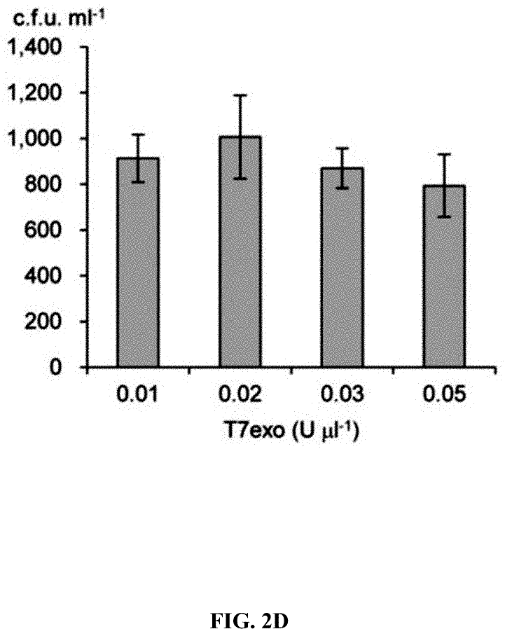

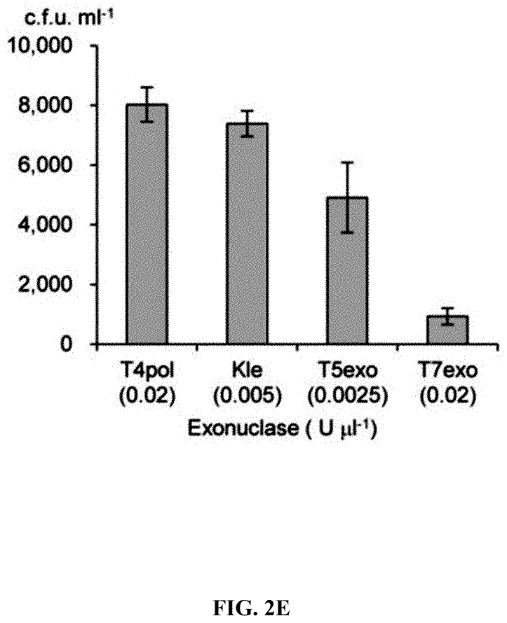

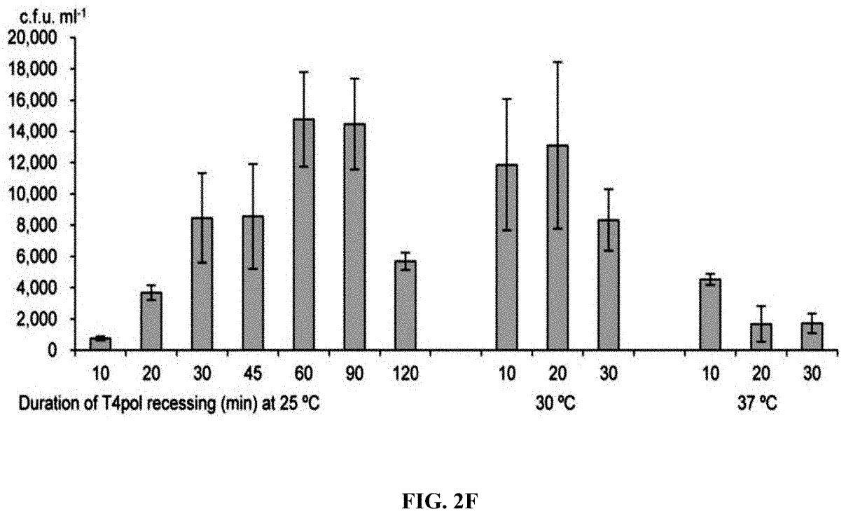

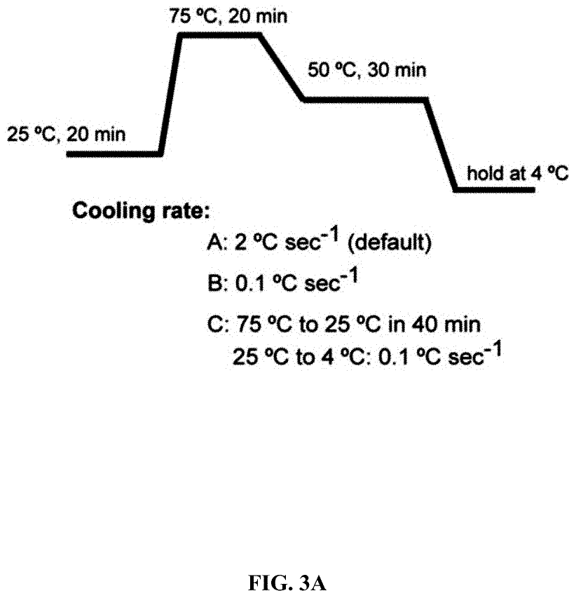

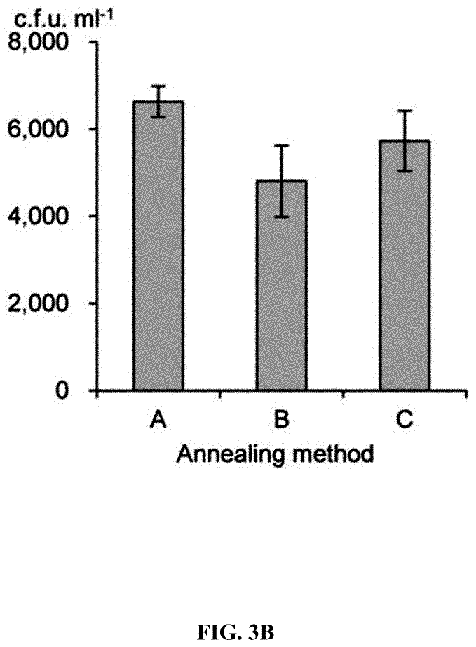

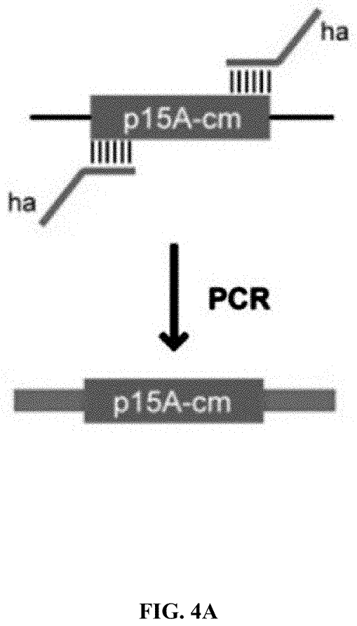

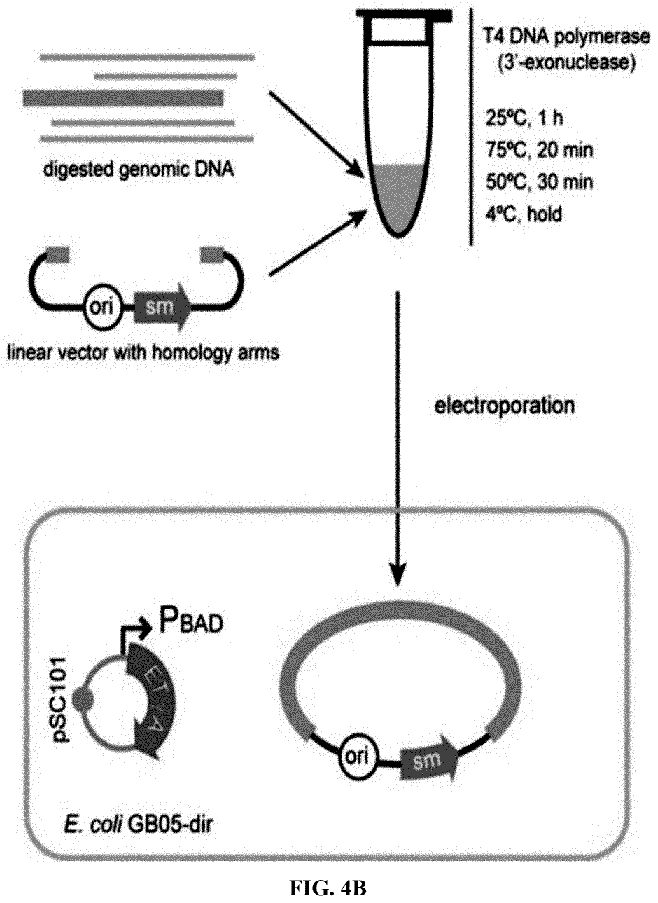

A series of exonucleases and annealing methods were tested by direct cloning of a 14-kb lux gene cluster of Photobacterium phosphoreum ANT-2200 (FIG. 1A). In this cloning experiment, 10 μg of ANT-2200 genomic DNA digested using BamHI and KpnI is mixed with 200 ng of 2.2-kb p15A-cm linear vector. The linear vector has a sequence at both ends that is identical to the ends (homologous box) of the genomic fragment containing the lux gene cluster produced by restriction digestion. The linear vector is homologously recombined with the target DNA fragment through the homologous boxes at both ends, to form a final circular plasmid. The cloning vector and BamHI+KpnI digested genomic DNA were first treated in vitro with different exonuclease enzymes, and then the reaction product after in vitro treatment was transformed into E. coli expressing the RecE/RecT recombinase. The tested exonucleases include: T4 DNA Polymerase (T4pol), Klenow Fragment of DNA Polymerase I (Kle), T7 DNA Polymerase (T7pol), Exonuclease III (ExoIII); Phusion DNA polymerase (Phusion DNA polymerase; Phu), T5 Exonuclease (T5exo); T7 Exonuclease (T7exo) and lambda exonuclease (λexo) (FIG. 2A). The results showed that T4pol, Kle, T5exo and T7exo significantly improves the efficiency of direct cloning (FIGS. 2A-2E). The annealing rate after exonuclease digestion have no effect on cloning efficiency, so a default cooling rate (2° C. s⁻¹) is chosen in the Eppendorf MC nexus PCR machine (FIGS. 3A-3B). The effect of the concentration, reaction temperature and reaction time of the T4pol on direct cloning efficiency are tested (FIG. 1C, d and FIG. 2F). The efficiency of the direct cloning using the full-length RecE/RecT alone, T4pol in vitro annealing alone, and combining T4pol in vitro annealing with full length RecE/RecT were compared (FIG. 1F). When RecE/RecT is used alone, the 14-kb lux gene cluster is directly cloned from the chromosome of the luminescent bacterium to the p15A vector with a high accuracy (427), but the direct cloning assembling the reaction products after T4pol in vitro assembly into a standard engineering E. coli, is much more efficient (427 vs 4,880). The results showed that: (1) E. coli endogenous DNA repair system can skillfully close the plasmid backbone; (2) T4pol is highly efficient in in vitro assembly (4880); (3) ExoCET technique that combines T4pol in vitro assembly with RecE/RecT intracellular recombination is more efficient than either technique alone (32500).

### Example 2

### Effect of Homology Boxes on Cloning Efficiency

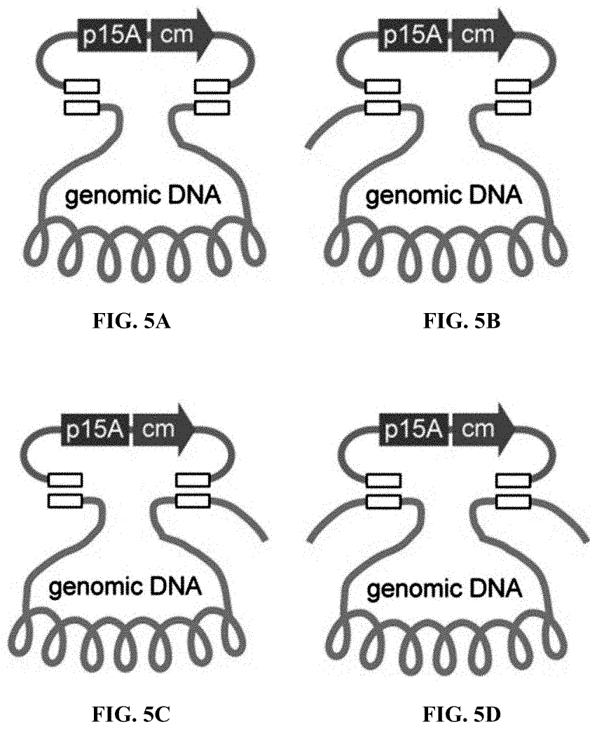

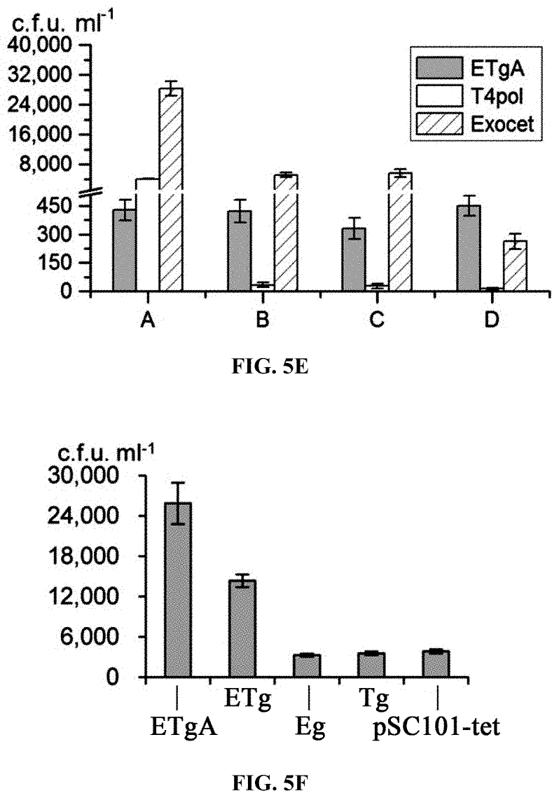

The longer the homology box, the higher the cloning efficiency (FIG. 1B). 80 bp homology box was placed at one end of the cloning vector at a position of 1-kb inside the gene cluster, or both ends at a position of 1-kb inside the gene cluster, and then compared the efficiency of ExoCET with the efficiency of T4pol and RecET (FIG. 5B). When both homologous boxes of the vector were at the very end, the same results were obtained as before. However, when one or two homology boxes were placed inside 1-kb of the gene cluster, the cloning efficiency of treatment with T4pol alone was very low, indicating that the annealing after T4pol exonuclease treatment depended on complementary pairing of the terminal DNA sequence. It was worth noting that the efficiency of RecET recombination had little to do with the position of the homology box. The experimental results obtained when one homology box was at the end and the other homology box was inside were thought-provoking. When there was only one terminal homology box between the vector and the genomic DNA fragment, the efficiency of ExoCET was 12 times than RecET, which indicated that T4pol in vitro treatment enabled two DNA molecules to be efficiently joined through annealing one end before the electroporation and the RecET recombination. The above data indicated that T4pol could only act on the terminal homology box, and the recombination of the internal homology box required the action of RecET. Therefore, the main contribution of T4pol to ExoCET was to increase their co-transformation efficiency by annealing one end of two linear DNA molecules. RecET could then be used to facilitate reorganization at the other end. In the cloning experiment of the 14-kb lux gene cluster, when both homology boxes were located at the very end of the target digested genomic fragment, the cloning efficiency of ExoCET was 6-8 times than T4pol (FIGS. 1F and 5B), which indicated that most of the in vitro assembled products had only one end joint together (>85%), and the recombinant plasmid produced by RecET after co-transformation of the in vitro assembled cyclic product and two linear DNA molecules contributed little to the high efficiency of ExoCET. When designing an ExoCET direct cloning experiment, the position of the homology box position was very important for cloning efficiency. To achieve the highest cloning efficiency, both homology boxes were placed at the very end of the target DNA fragment. In fact, in order to exert the effect of T4pol exonuclease and annealing in vitro, at least one homology box was placed at the end. However, another homology box could be placed inside the target DNA fragment because RecE/RecT could localize it and recombine it. This was very advantageous for the use of direct cloning to construct expression vectors, since one of the homology boxes could be placed at the very end of the 3′ end of the target fragment, and the 5′ end of the target gene could be placed directly under the control of the promoter and ribosome binding sites using internal homology boxes.

### Example 3

### RecE and RecT were Both Required for ExoCET

Since RecT is a single-stranded DNA annealing protein, RecT may anneal the single-stranded DNA region produced by T4pol (3′ exonuclease), so RecE may not be required in the ExoCET technique system. In order to verify this conjecture, the T4pol-treated DNA substrate was transformed into E. coli cell expressing RecT and Redγ (pSC101-Tg) and not expressing RecE, and found no interaction between RecT and T4pol. Therefore, both RecE and RecT were required for ExoCET (FIG. 5C). RecA had a certain improvement in the efficiency of direct cloning.

### Example 4

### Verification of Direct Cloning of Large DNA Fragments

To verify the superiority of ExoCET technique, some experiments that were difficult to be finished with RecET technique were performed. There are two large gene clusters on the genome of Photorhabdus luminescens: 37.5-kb plu3535-3532 and 52.6-kb plu2670. It was very difficult to directly clone these two gene clusters with RecET technique, and the efficiencies were only 2/12 and 0/48, respectively. While using ExoCET technique achieved correct rates of 10/12 and 11/17, respectively (Table 2).

TABLE 2
Large fragments of genomic DNA cloned directly from bacteria,
mammalian cells, and human blood using ExoCET

| | | | | | | | Correct |
|---|---|---|---|---|---|---|---|
| | | | Genomic | | | Number of | number/ |
| | | Genome | restriction | Size | | colonies | Detection |
| Target gene | Source | (Mb) | enzyme | (kb) | Vector | (/mL) | number |
| plu3535-3532 | (Photorhabdus | 5.69 | XbaI | 38 | pBAC2015 | 1815 ± 132 | 10/12 |
| | luminescens) | | | | | | |
| | DSM15139 | | | | | | |
| plu2670 | (Photorhabdus | 5.69 | XbaI + XmaI | 53 | p15A | 1152 ± 211 | 11/17 |
| | luminescens) | | | | | | |
| | DSM15139 | | | | | | |
| Salinomycin | (Streptomyces | 8.38 | EcoRV | 106 | pBeloBAC11 | 425 ± 91 | 2/24 |
| gene cluster | albus) | | | | | | |
| | DSM41398 | | | | | | |
| Salinomycin | (Streptomyces | 8.38 | Cas9 | 106 | pBeloBAC11 | 260 ± 14 | 1/24 |
| gene cluster | albus) | | | | | | |
| | DSM41398 | | | | | | |
| Prkar1a | Mouse melanoma | 2800.06 | HpaI | 8 | p15A | 205 ± 17 | 10/12 |
| | B16 cells | | | | | | |
| Dpy30 | Mouse melanoma | 2800.06 | BamHI + | 8.7 | p15A | 273 ± 18 | 9/12 |
| | B16 cells | | KpnI | | | | |
| Wnt4 | Mouse melanoma | 2800.06 | SwaI | 45 | p15A | 76 ± 16 | 8/25 |
| | B16 cells | | | | | | |
| Lmbr1l-Tuba1a | Mouse melanoma | 2800.06 | SwaI | 53 | p15A | 52 ± 6 | 1/12 |
| | B16 cells | | | | | | |
| DPY30 | Human | 3221.49 | SpeI | 9.1 | p15A | 40 ± 10 | 17/24 |
| | embryonic | | | | | | |
| | kidney 293T cells | | | | | | |
| DPY30 | Human blood | 3221.49 | SpeI | 9.1 | p15A | 45 ± 2 | 5/24 |
| IGFLR1-LIN37 | Human blood | 3221.49 | NdeI + BstZ17I | 14 | p15A | 320 ± 67 | 9/48 |
| IGFLR1-ARHGAP33 | Human blood | 3221.49 | BstZ17I | 41 | p15A | 275 ± 76 | 5/48 |
| ZBTB32-LIN37 | Human blood | 3221.49 | NdeI | 45 | p15A | 115 ± 35 | 2/48 |
| Oct4-Venus | Mouse R1 | 2800.06 | EcoRV +PacI | 9.6 | p15A | 34 ± 1 | 9/36 |
| | embryonic stem | | | | | | |
| | cell | | | | | | |
| Nanog-Cherry | Mouse R1 | 2800.06 | NdeI | 13 | p15A | 49 ± 12 | 17/54 |
| | embryonic stem | | | | | | |
| | cell | | | | | | |
| Gata2-Venus | Mouse R1 | 2800.06 | BstZ17I | 16.8 | p15A | 212 ± 27 | 5/45 |
| | embryonic stem | | | | | | |
| | cell | | | | | | |
| Mll4(1) | Mouse R1 | 2800.06 | SspI + SpeI | 17.1 | p15A | 127 ± 38 | 7 + 3/24 |
| | embryonic stem | | | | | | |
| | cell | | | | | | |
| Mll4(2) | Mouse R1 | | | | | 323 ± 65 | 2 + 2/36 |
| | embryonic stem | | | | | | |
| | cell | | | | | | |
| Mll4(3) | Mouse R1 | | | | | 142 ± 27 | 6 + 9/72 |
| | embryonic stem | | | | | | |
| | cell | | | | | | |
| Mll4(4) | Mouse R1 | | | | | 483 ± 91 | 3 + 5/36 |
| | embryonic stem | | | | | | |
| | cell | | | | | | |

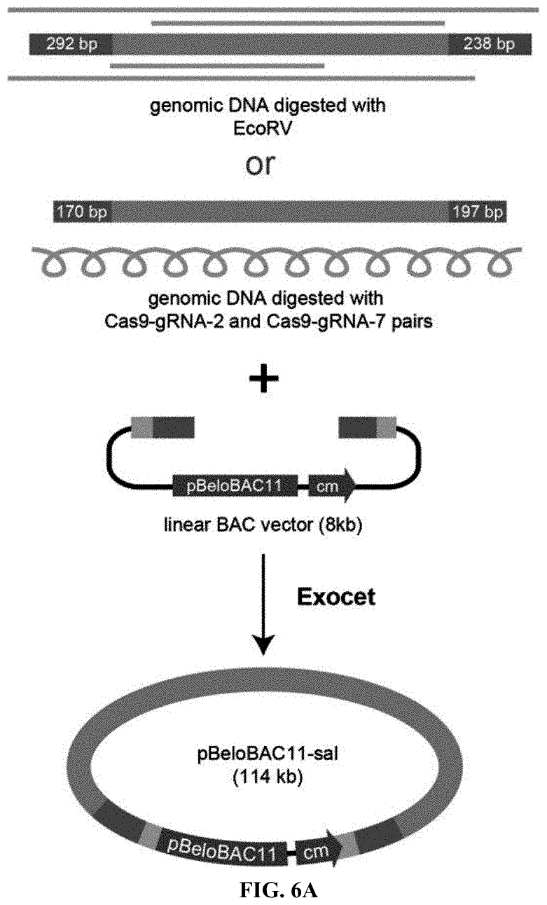

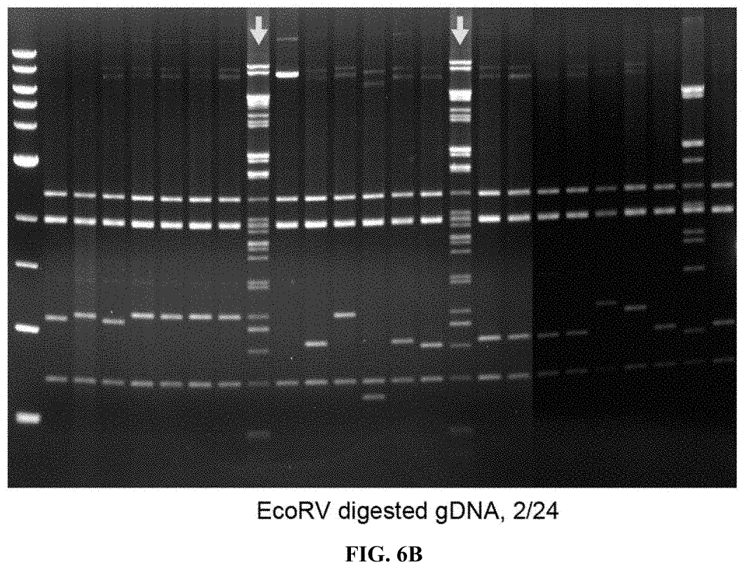

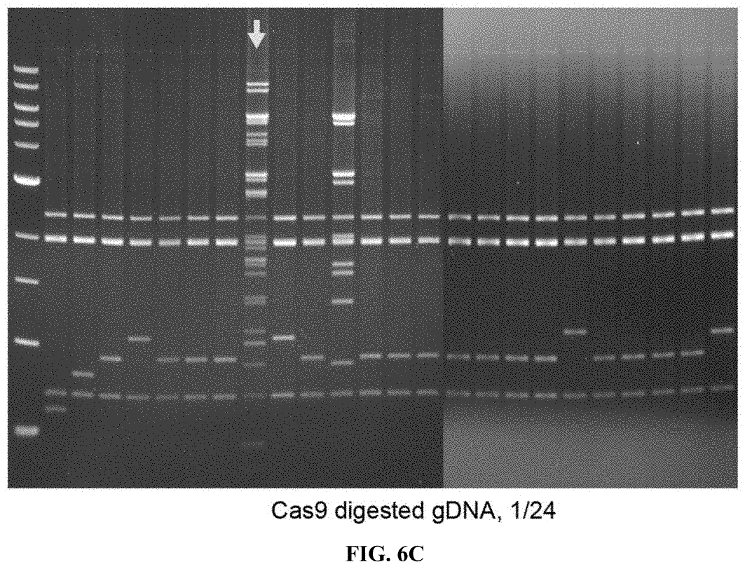

Previously, the attempt to directly clone the 106-kb salinomycin gene cluster from Streptomyces albicans genome was failed. So, the gene cluster was divided into three fragments which were cloned step by step and then integrated to form a complete gene cluster. However, through ExoCET, the 106-kb salinomycin gene cluster can be directly cloned into the BAC vector by using a BAC vector with a homology box and EcoRV digested genomic DNA, and obtained the correct rate of 2/24 (Table 2 and FIGS. 6A-6C). Since there was an EcoRV restriction site on each side of the 106-kb salinomycin gene cluster, the gene cluster can be released from the chromosome and cloned into the vector. When cloning large DNA fragments, it was difficult to find suitable restriction sites on both sides of the target DNA fragment, but the use of programmable nucleases could eliminate the restriction on restriction sites, especially is an RNA-mediated endonuclease-Cas9. Cas9 was used to release the 106-kb salinomycin gene cluster from the chromosome where very closed to the EcoRV, and then cloned the same 106-kb DNA fragment into the BAC vector using the same BAC vector. A similar cloning efficiency was finally obtained (Table 2 and FIGS. 6A-6C). Therefore, ExoCET had a significantly superior performance in direct cloning of large DNA fragments compared to RecET.

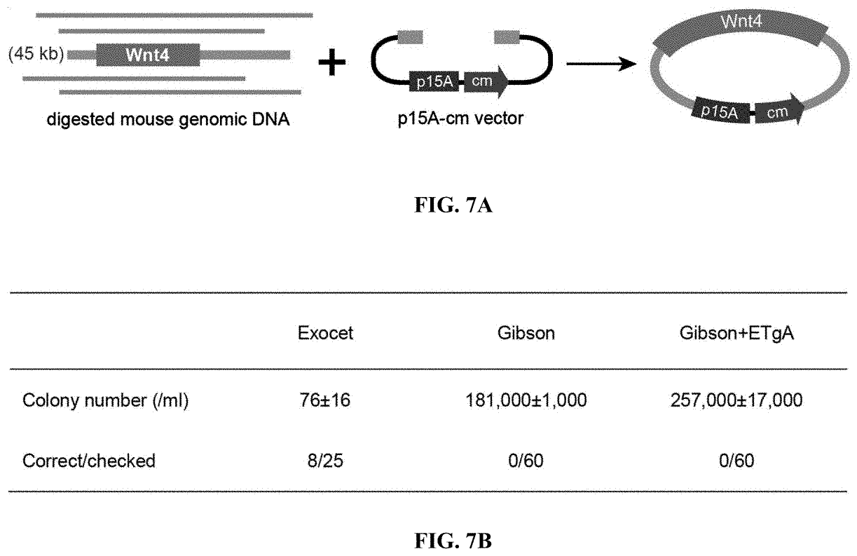

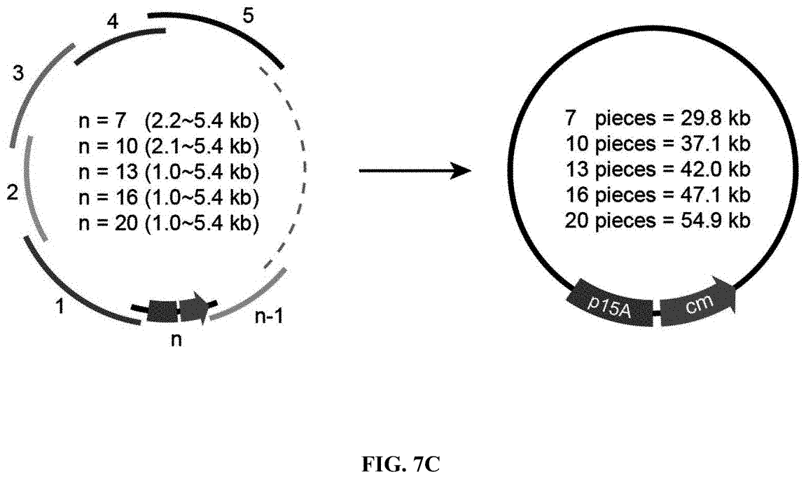

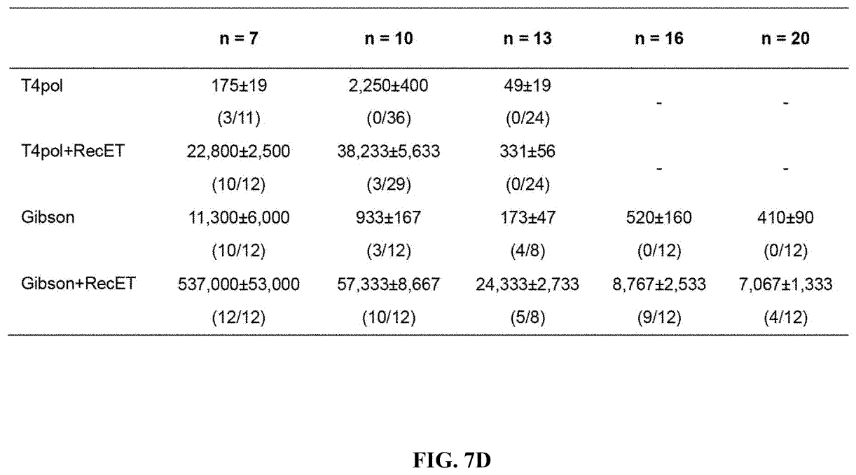

Next, it is tested whether the efficiency of ExoCET could meet the requirement of directly cloning large DNA fragments from mammalian genome. SwaI was used to release a 45-kb fragment containing the Wnt4 gene from the mouse genome (FIG. 7A), and the correct rate of 8/25 was obtained through ExoCET (FIG. 7B). The use of Gibson assembly was tested to clone this DNA fragment. Gibson assembly uses T5exo, Phusion DNA polymerase and Taq DNA ligase to assemble DNA molecules with homology boxes between each other. A large number of colonies (181,000 and 257,000) was obtained by transforming the Gibson assembled DNA product into arabinose-induced and non-induced E. coli GB05-dir containing pSC101-BAD-ETgA-tet. And 60 colonies were detected and no correct clone was obtained (FIG. 7B), and all were p15A empty vectors of self-circularization.

### Example 5

### Assembly of DNA Fragments Using ExoCET

Gibson was a method for multiple fragment DNA assembly, and ExoCET and Gibson through some DNA multiple fragment assembly experiments (FIG. 7C) was compared. These DNA fragments were amplified by PCR and had a 40-bp homology box at the end. The efficiency of ExoCET and Gibson assembly was good in the 7-fragment and 10-fragment assembly experiments. When Gibson's in vitro assembled products were transformed into E. coli cell expressing RecE, RecT, Redγ and RecA (Gibson and ETgA), assembly efficiency and accuracy were significantly improved (FIG. 7D). ExoCET couldn't assemble more than 13 DNA fragments, Gibson couldn't assemble more than 16 DNA fragments, while Gibson and ETgA could assemble at least 20 DNA fragments into a 54.9-kb plasmid. Therefore, combining in vitro assembly with RecET recombination, the advantages for DNA assembly were obvious.

### Example 6

### Construction of Haplotype Isogenic Targeting Vector Using ExoCET

ExoCET could also be used to directly clone DNA fragments from mammalian genomes including blood, disease-associated cell lines, etc. to facilitate haplotype studies of SNPs and to rapidly construct haplotype syngeneic (HIT) targeting vectors for targeting of nuclease-mediated human stem cells. The importance of human stem cells isolated from patients, cord blood or somatic cell reprogramming in biomedical research had received more and more attention. The research on the precise modification of stem cell genome had also received widespread attention. Transforming the human genome was more challenging than structuring the genome of experimental mice because human genetic diversity was complex. The importance of isogenicity (sequence similarity) for homologous recombination was realized many years ago when people use mouse embryonic stem cells for gene targeting.

Unlike the method of amplifying homology boxes from the genome by PCR, ExoCET was not limited by fragment size, did not introduce mutations, and was capable of maintaining a DNA haplotype. Furthermore, the ends of the homology boxes could also be selected according to the manner of genotyping (such as Southern blotting or joining PCR), so the length of the homology boxes could be optimized. ExoCET therefore offers advantages for individualized genomic surgery, especially when combined with CRISPR/Cas9.

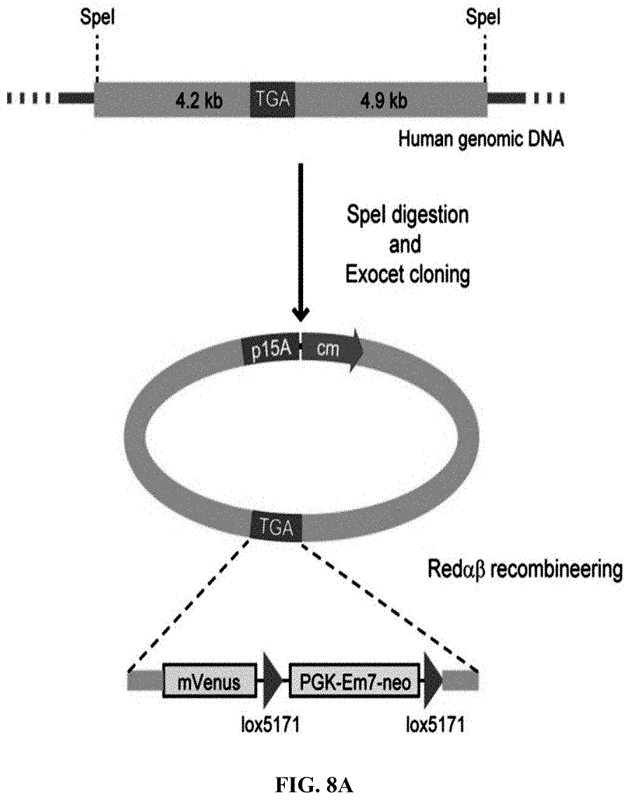

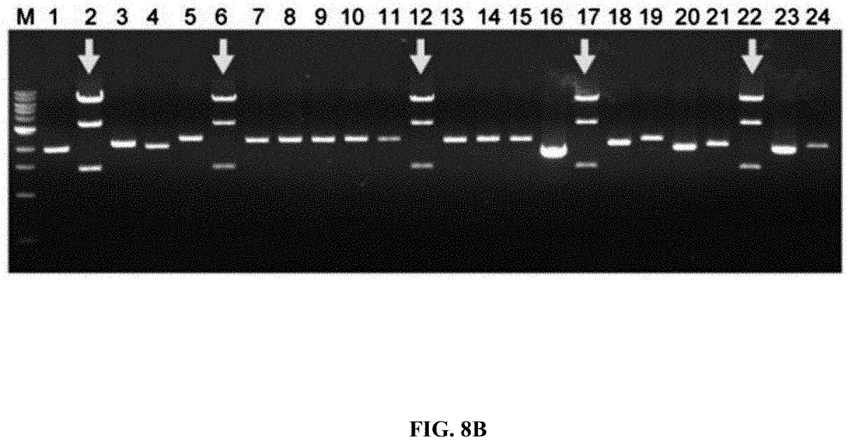

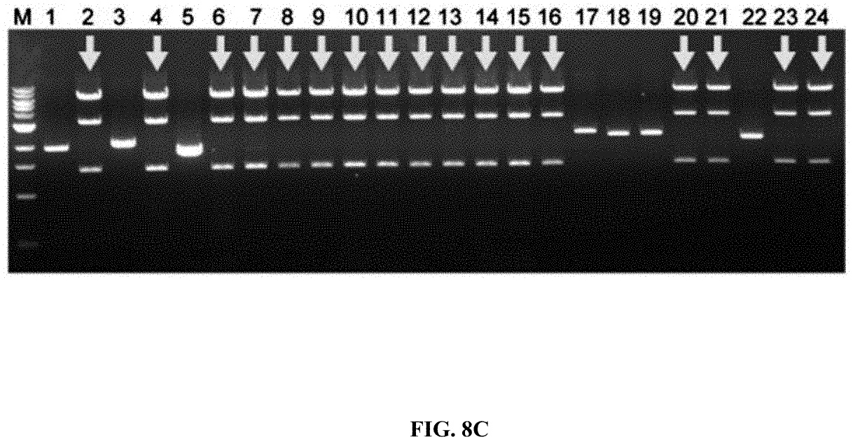

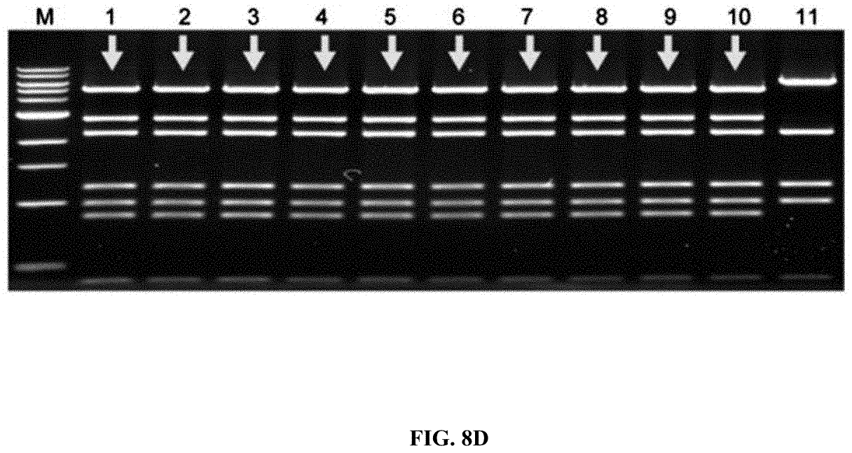

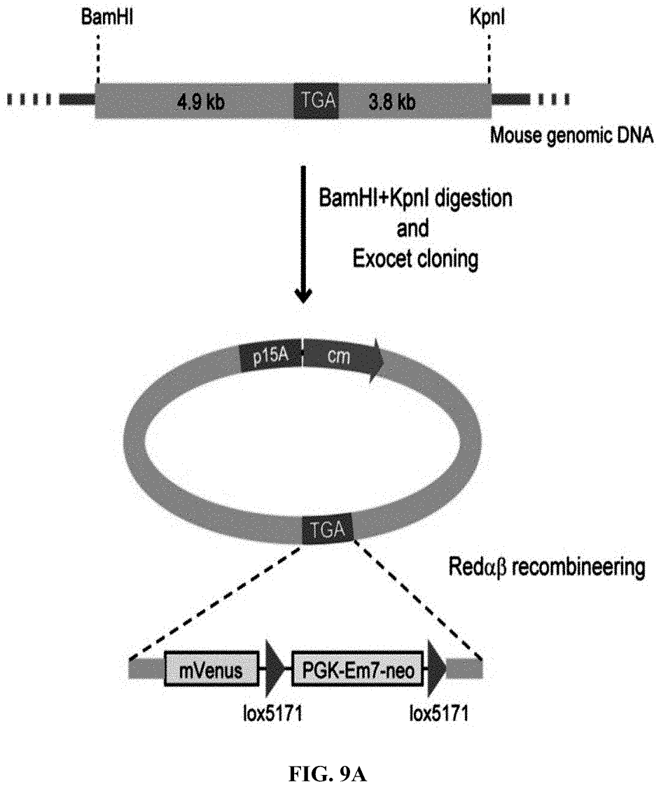

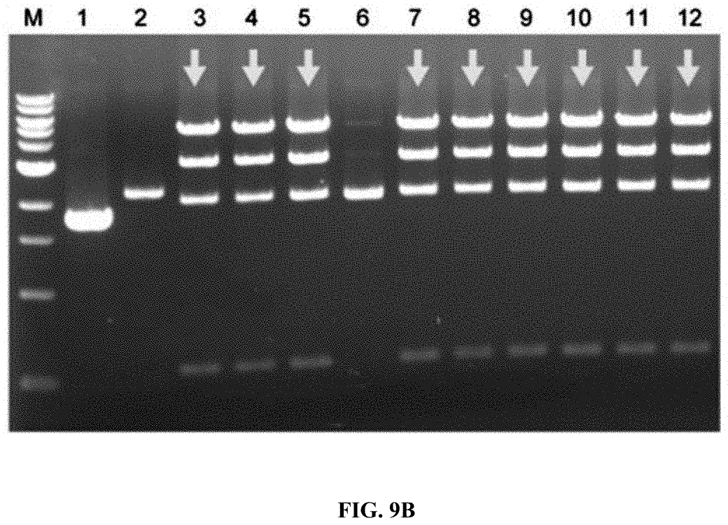

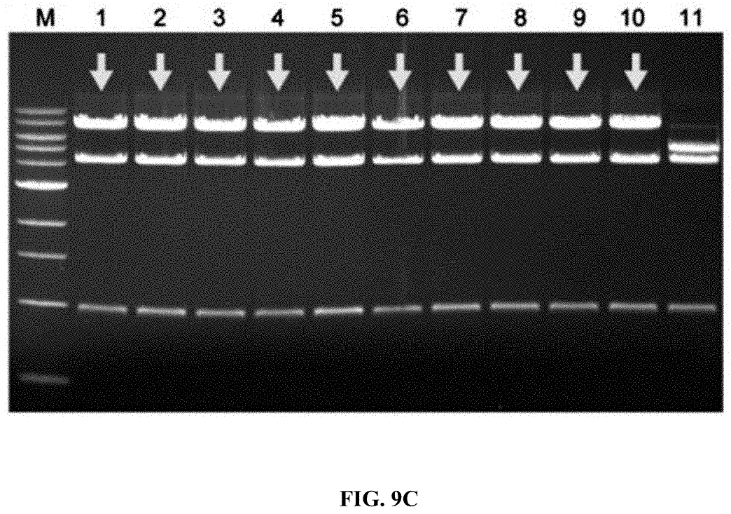

ExoCET was used to construct isogenic targeting vectors to modify mammalian genomes. Given the experience in mouse embryonic stem cell research, one purpose was to clone a 5 to 10-kb DNA fragment directly from human or mouse genome as an isogenic homology box (FIG. 8A and FIG. 9A). It is noteworthy that these DNA fragments were not only the same gene but also maintain the polymorphic haplotype, so it was called HIT (haplotype isogenic targeting) vector. 8-9-kb DNA fragments was directly cloned from the human genomes (in vitro cultured cell lines and human blood) and mouse (in vitro cultured cell lines) by ExoCET (FIG. 8B and FIG. 9B). Selection markers and other functional elements were then inserted into the HIT vector by Redαβ recombineering (FIG. 8C and FIG. 9C).

### Example 7

### Genotyping Mammalian Cells Using ExoCET

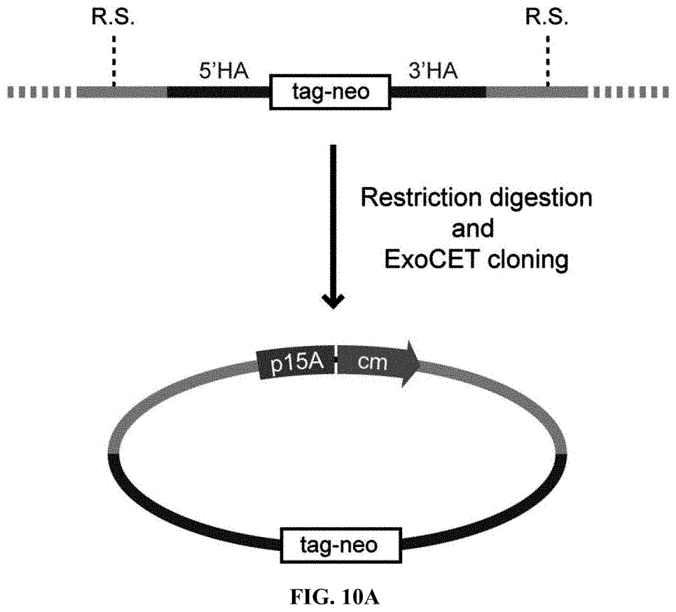

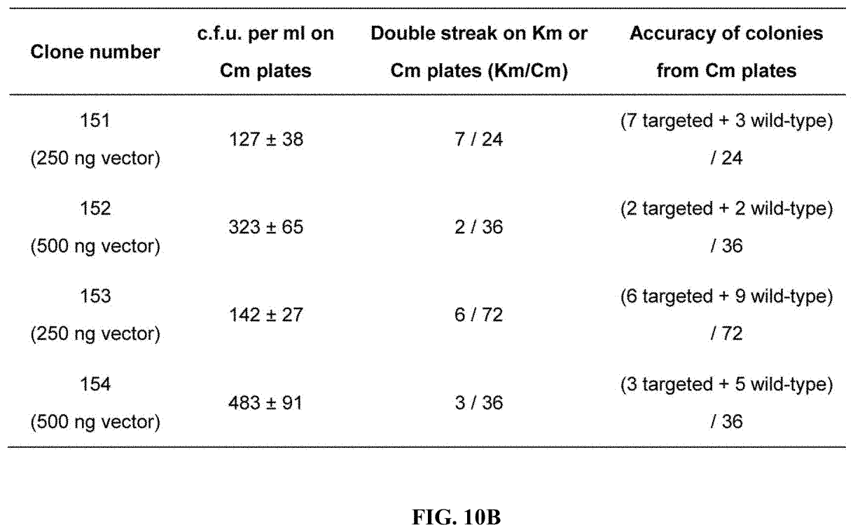

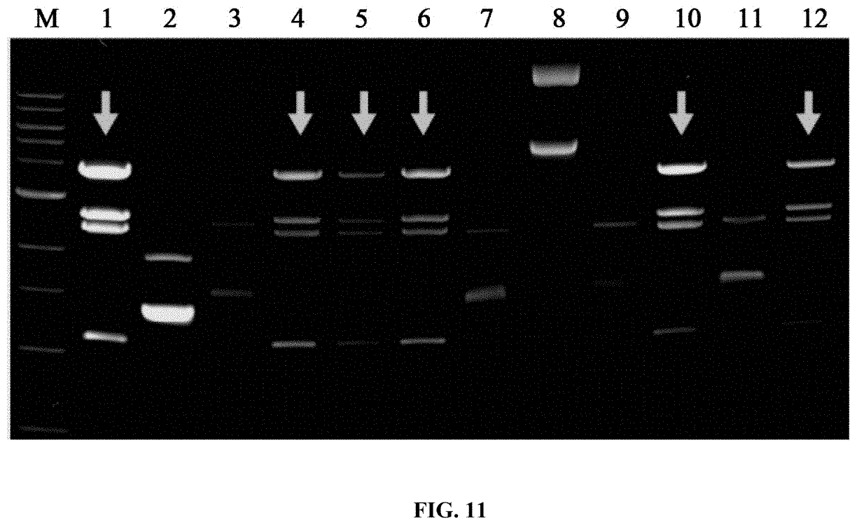

ExoCET could also be used as the most reliable method for genotyping a modified genome, while Southern blotting and joining PCR could produce false positive signals. Since long range PCR was prone to false positive signals in mammalian genotyping studies, it is need to confirm the Kmt2d-AID-neo-targeted mouse embryonic stem cells screened by long-segment PCR by Southern blotting. However, no probe was available. Therefore, a DNA fragment containing the entire targeting element was cloned from the genome of four possible Kmt2d-AID-neo-targeted mouse embryonic stem cells using the ExoCET method shown in FIG. 10A. The recombinant plasmid cloned by ExoCET was subjected to restriction analysis and DNA sequencing, and the results showed that the four cells were successfully targeted and were single-targeted (FIG. 10B), which was further testified by ExoCET using re-Oct4-Venus-neo, Nanog-Cherry-neo, Gata2-Venus-neo and Set1b-TC-neo target mouse embryonic stem cells (Table 3). These target cells previously verified by Southern blotting. These results indicated that there was no site restriction on ExoCET genotyping. Previously, it is failed to determine whether a Klf4-Venus-neo-targeted mouse embryonic stem cell was successfully targeted, because long-range PCR and Southern blotting had not obtained an exact signal. No DNA fragment having kanamycin resistance in the corresponding region on the genome was cloned by using ExoCET (Table 3). Restriction analysis of the cloned chloramphenicol-resistant plasmid found that 50% of them contained the wild-type DNA sequence (FIG. 11). Therefore, this cell was not correctly targeted.

TABLE 3
Experimental data of ExoCET genotyping

| | | | | | Correct rate |
|---|---|---|---|---|---|
| Mouse | | | | | of colonies |
| embryonic stem | | | | Kanamycin and | on the |
| cell | | | Number of | chloramphenicol | double-antiplate |
| (Amount of | Genomic | | colonies on | plates/ | of |
| transformed | restriction | Size | chloramphenicol | Double hyphen | kanamycin + |
| DNA) | enzyme | (kb) | plates (/mL) | (Km/Cm) | chloramphenicol* |
| Oct4-Venus-neo | EcoRV + | 9.6 | 34 ± 1 | 9/36 | 9/9 |
| #7 (R1) | PacI | | | | |
| (10 μg genomic | | | | | |
| DNA + 250 ng | | | | | |
| vector) | | | | | |
| Nanog-Cherry- | NdeI | 13 | 49 ± 12 | 17/54 | 7/17 ** |
| neo #18 (R1) | | | | | |
| (7.7 μg genomic | | | | | |
| DNA + 400 ng | | | | | |
| vector) | | | | | |
| Gata2-Venus-neo | BstZ17I | 16.8 | 212 ± 27 | 5/45 | 5/5 |
| #19 (GM8) | | | | | |
| (1.5 μg genomic | | | | | |
| DNA + 500 ng | | | | | |
| vector) | | | | | |
| Set 1b-TC-neo | AseI | 24 | 49 ± 8 | 1/36 | 1/1 |
| #4 (R1) | | | | | |
| (10 μg genomic | | | | | |
| DNA + 250 ng | | | | | |
| vector) | | | | | |
| Klf4-Venus-neo | AflII | 10.2 | 18 ± 4 | 0/36 | — |
| #9 (R1) | | | | | |
| (10 μg genomic | | | | | |
| DNA + 250 ng | | | | | |
| vector) | | | | | |

*in Table 3 represents after restriction analysis.
** in Table 3 means the remaining 10 were intramolecular recombination (containing 11 direct repeats greater than 40 bp in the cloned target sequence).

TABLE 4
The amount of Oct4-Venus-neo targeted mouse embryonic stem
cell genomic DNA required for optimizing ExoCET genotyping

| | Number of colonies on | |
|---|---|---|
| | kanamycin and | |
| | chloramphenicol | Correct number/ |
| Vector | double-resistance plate (/mL) | detection number* |
| 500 ng of genomic DNA digested with EcoRV and PacI was | | |
| mixed with the amount of p15A-cm vector shown below | | |
| 500 ng | 12 | 3/3, 100% |
| 1000 ng | 10 | 3/3, 100% |
| 2000 ng | 8 | 3/3, 100% |
| 1000 ng of genomic DNA digested with EcoRV and PacI was | | |
| mixed with the amount of p15A-cm vector shown below | | |
| 500 ng | 10 | 3/3, 100% |
| 1000 ng | 10 | 3/3, 100% |
| 2000 ng | 10 | 3/3, 100% |

*in Table 4 represents after restriction analysis.

ExoCET genotyping did not produce a false positive signal compared to long range PCR. Compared to Southern blotting, ExoCET genotyping is simpler and did not require cumbersome screening of hybridization probes. In ExoCET genotyping, restriction enzyme sites for the release of intact targeting elements were easily available, and in the case of well-prepared genomes, genotyping results were obtained in three days. More importantly, ExoCET never produced a false positive signal. Since the targeting element had a selection marker, as long as 500 ng of restriction enzyme genomic DNA was sufficient to obtain better cloning efficiency (Table 4). To increase the throughput of ExoCET genotyping, cells cultured in 96-well plates can be used.

### Example 8

### ExoCET Cloning Technique Applied to Metagenomic Samples

Functional analysis of whole genome sequencing results requires a simple and rapid method of expression vector construction. According to the method of the disclosure, a DNA fragment of up to 50-kb can be cloned from a 3.0×10⁹-bp genome. To this end, 1 ng of P. phosphoreum genomic DNA was diluted and added to 10 μg of Bacillus subtilis genomic DNA to mimic the metagenomics. The experiment successfully cloned the 14-kb lux gene cluster by ExoCET and obtained considerable efficiency (Table 5). Environmental samples usually contained more than 10⁴ species, so the results show ExoCET cloning technique can be applied to metagenomic samples.

TABLE 5
The 14 kb lux gene cluster was cloned directly from the
diluted P. phosphoreum genome using ExoCET

| P. phosphoreum | B. subtilis | | Number of | Correct number/ |
|---|---|---|---|---|
| (ng) | (μg) | | colonies | Detection |
| (BamHI + KpnI) | (BamHI) | Vectors | (/mL) | number |
| 10 | 10 | p15A-cm | 200 ± 2 | 7/12 |
| 5 | 10 | p15A-cm | 142 ± 22 | 5/12 |
| 2 | 10 | p15A-cm | 102 ± 8 | 2/12 |
| 1 | 10 | p15A-cm | 104 ± 18 | 2/24 |

In this disclosure, the genomic DNA and the cloning vector are cleaved with exonuclease in vitro, and the reaction product in vitro was then homologously recombined in the presence of RecET recombinase to establish the ExoCET cloning technique. ExoCET technique can clone DNA fragments larger than 100-kb directly from bacterial genome, and clone DNA fragments larger than 50-kb from mammalian cells and human blood. ExoCET technique is also capable of efficiently assembling at least twenty DNA fragments to form a complete plasmid. Like the RecET direct cloning technique, ExoCET presents advantages over PCR for amplification of DNA because it has a much higher fidelity, is not limited in size and does not scramble haplotypes. The target DNA is directly cloned into a plasmid vector to facilitate expression studies. In addition, ExoCET is more efficient than Gibson assembly because Gibson relies on circular DNA molecules produced by in vitro assembly. Moreover, due to the self-circularization of the empty vector, Gibson assembly may produce a very serious background in the process of direct cloning.

Through the ability to selectively acquire large DNA segments from complex genomic preparations including blood, ExoCET also presents options for diagnostics and pathology tests such as directed sequence acquisition for personal medicine or the isolation of DNA viruses from patient materials. ExoCET will have broad applications in functional and comparative genomics, especially for direct cloning of biosynthetic pathways from prokaryotes or assembling multiple DNA molecules for synthetic biology.

It will be obvious to those skilled in the art that changes and modifications can be made, and therefore, the aim in the appended claims is to cover all such changes and modifications.

What is claimed is:

1. A method, comprising: cleaving and annealing two or more target nucleic acid molecules with a first exonuclease, and recombining the two or more target nucleic acid molecules in the presence of a second exonuclease and an annealing protein, wherein recombined target nucleic acid molecules share at least one homologous sequence.

2. The method of claim 1, wherein the at least one homologous sequence is in or at one end of each of the two or more target nucleic acid molecules.

3. The method of claim 2, wherein the at least one homologous sequence comprises at least 6 nucleotides.

4. The method of claim 1, wherein the first exonuclease is selected from the group consisting of T4 DNA polymerase, Klenow fragment of DNA polymerase I, T5 exonuclease, and T7 exonuclease.

5. The method of claim 1, wherein cleaving and annealing two or more target nucleic acid molecules further comprises addition of a DNA polymerase, dNTPs and a DNA ligase.

6. The method of claim 1, wherein the second exonuclease is RecE.

7. The method of claim 1, wherein the annealing protein is selected from RecA, RAD51, Redβ, RecT, Pluβ and RAD52.

8. The method of claim 1, wherein recombining the two or more target nucleic acid molecules is carried out in vitro, in a yeast cell, or a bacterial cell.

9. The method of claim 8, wherein the host cell expresses an exonuclease, an annealing protein, or a combination thereof.

10. The method of claim 9, wherein the host cell is E. coli cell expressing full length RecE and/or RecT.

11. The method of claim 9, wherein the host cell expresses the exonuclease and the annealing protein on a plasmid vector and/or a chromosome.

12. The method of claim 1, wherein the two or more target nucleic acid molecules are a linear DNA segment selected from a DNA fragment digested by endonuclease, a DNA fragment amplified by PCR, a genomic DNA fragment, a member of cDNA library, a fragment derived from bacterial artificial chromosomes (BACs), and a fragment of cloning vectors.

13. A method, comprising: cleaving and annealing two or more nucleic acid molecules with a first exonuclease, and recombining the two or more nucleic acid molecules in the presence of a second exonuclease and an annealing protein, wherein each nucleic acid molecule shares at least one homologous sequence with an adjacent nucleic acid molecule in a resulting assembly product.

14. The method of claim 13, wherein the at least one homologous sequence is in or at one end of each of the two or more nucleic acid molecules.

15. The method of claim 14, wherein the at least one homologous sequence comprises at least 6 nucleotides.

16. The method of claim 13, wherein the first exonuclease is selected from the group consisting of T4 DNA polymerase, Klenow fragment of DNA polymerase I, T5 exonuclease, and T7 exonuclease.

17. The method of claim 13, wherein cleaving and annealing two or more nucleic acid molecules further comprises addition of a DNA polymerase, dNTPs and a DNA ligase.

18. The method of claim 13, wherein the second exonuclease is RecE.

19. The method of claim 13, wherein the annealing protein is selected from RecA, RAD51, Redβ, RecT, Pluβ and RAD52.

20. The method of claim 13, wherein recombining the two or more nucleic acid molecules is carried out in vitro, in a yeast cell, or a bacterial cell.

21. The method of claim 20, wherein the host cell expresses an exonuclease, an annealing protein, or a combination thereof.

22. The method of claim 21, wherein the host cell is E. coli cell expressing full length RecE and/or RecT.

23. The method of claim 21, wherein the host cell expresses the exonuclease and the annealing protein on a plasmid vector and/or a chromosome.

24. The method of claim 13, wherein the two or more nucleic acid molecules are a linear DNA segment selected from a DNA fragment digested by endonuclease, a DNA fragment amplified by PCR, a genomic DNA fragment, a member of cDNA library, a fragment derived from bacterial artificial chromosomes (BACs), and a fragment of cloning vectors.

25. A method, comprising: cleaving and annealing a linear cloning vector and a mixture of genomic DNA fragments with a first exonuclease, and recombining the linear cloning vector and a target DNA fragment of the mixture of genomic DNA fragments, wherein the linear cloning vector shares at least one homologous sequence with the target DNA fragment of the mixture of genomic DNA fragments.

26. The method of claim 25, wherein the at least one homologous sequence is in or at one end of each of the two or more target nucleic acid molecules.

27. The method of claim 26, wherein the at least one homologous sequence comprises at least 6 nucleotides.

28. The method of claim 25, wherein the first exonuclease is selected from the group consisting of T4 DNA polymerase, Klenow fragment of DNA polymerase I, T5 exonuclease, and T7 exonuclease.

29. The method of claim 25, wherein cleaving and annealing a linear cloning vector and a mixture of genomic DNA fragments further comprises addition of a DNA polymerase, dNTPs and a DNA ligase.

30. The method of claim 25, wherein the second exonuclease is RecE.

31. The method of claim 25, wherein the annealing protein is selected from RecA, RAD51, Redβ, RecT, Pluβ and RAD52.

32. The method of claim 25, wherein recombining the linear cloning vector and a target DNA fragment of the mixture of genomic DNA fragments is carried out in vitro, in a yeast cell, or a bacterial cell.

33. The method of claim 32, wherein the host cell expresses an exonuclease, an annealing protein, or a combination thereof.

34. The method of claim 33, wherein the host cell is E. coli cell expressing full length RecE and/or RecT.

35. The method of claim 33, wherein the host cell expresses the exonuclease and the annealing protein on a plasmid vector and/or a chromosome.

36. The method of claim 25, wherein the target DNA fragment is a linear DNA segment selected from a DNA fragment digested by endonuclease, a DNA fragment amplified by PCR, a genomic DNA fragment, a member of cDNA library, a fragment derived from bacterial artificial chromosomes (BACs), and a fragment of cloning vectors.

37. A method of DNA synthesis, construction of a targeting vector, genotyping, and/or preparation of a kit using the method of claim 1.

38. A method of DNA synthesis, construction of a targeting vector, genotyping, and/or preparation of a kit using the method of claim 2.

39. A method of DNA synthesis, construction of a targeting vector, genotyping, and/or preparation of a kit using the method of claim 3.

40. A method of DNA synthesis, construction of a targeting vector, genotyping, and/or preparation of a kit using the method of claim 12.

41. A method of DNA synthesis and/or preparation of a kit using the method of claim 13.

42. A method of DNA synthesis and/or preparation of a kit using the method of claim 14.

43. A method of DNA synthesis and/or preparation of a kit using the method of claim 15.

44. A method of DNA synthesis and/or preparation of a kit using the method of claim 24.

45. A method of construction of a targeting vector, genotyping, and/or preparation of a kit using the method of claim 25.

46. A method of construction of a targeting vector, genotyping, and/or preparation of a kit using the method of claim 26.

47. A method of construction of a targeting vector, genotyping, and/or preparation of a kit using the method of claim 27.

48. A method of construction of a targeting vector, genotyping, and/or preparation of a kit using the method of claim 36.
